(19) United States
(12) Patent Application Publication
(10) Pub. No.: US 2024/0233865 A1
(43) Pub. Date: Jul. 11, 2024

(54) METHODS FOR PREDICTING AND EVALUATING FOOD FUNCTIONS IN PROTEINS AND COMPOSITIONS

(71) Applicant: Prose Foods, Inc., San Carlos, CA (US)

(72) Inventors: Hermann Broder Schmidt, Redwood City, CA (US); Daniel Griffith, St. Louis, MO (US)

(21) Appl. No.: 18/401,657

(22) Filed: Jan. 1, 2024

Related U.S. Application Data
(60) Provisional application No. 63/437,489, filed on Jan. 6, 2023.

(51) Int. Cl. G16B 20/00; A23L 33/17

(52) U.S. Cl. CPC G16B 20/00; A23L 33/17

(57) ABSTRACT

A method of predicting at least one target food function of a candidate protein comprises providing an amino acid sequence for the candidate protein; computer processing, by at least one processor executing a trained computer model, the amino acid sequence of the candidate protein to predict a set of candidate amino acid sequences having intrinsic disorder, wherein the intrinsic disorder may comprise a lack of stable secondary structure along at least about 10% of the length of an individual candidate amino acid sequence; and computer processing, by at least one processor executing a trained computer model, the set of candidate amino acid sequences to generate an output that may be indicative of the predicted target food function or target food functions of the candidate protein. Compositions and food products comprising the candidate protein are also disclosed.

### INCORPORATION BY REFERENCE OF A SEQUENCE LISTING

A sequence listing is provided herewith, the content being the electronically submitted Sequence Listing (name: “P1321US_Sequence Listing_2023-12-22.xml”; size: 56,131 bytes; and date of creation: Jan. 1, 1024), is herein incorporated by reference in its entirety.

### TECHNICAL FIELD

The embodiments disclosed herein relate to alternative protein food products and, in particular, to systems and methods for evaluating food functions of candidate proteins.

### BACKGROUND

Agriculture is a significant contributor to anthropogenic global warming, and reducing agricultural emissions-largely methane and nitrous oxide—may play a significant role in climate change mitigation. The global food system is responsible for ˜21-37% of annual emissions, as commonly reported using the 100-year Global Warming Potential (more on this later). The composition of gases emitted by the food system does not reflect the overall global emissions balance, however, with agricultural activity generating around half of all anthropogenic methane emissions and around three-quarters of anthropogenic N2O (Lynch et al., Front. Sustain. Food Syst. 4:518039, which is incorporated by reference herein).

Despite animal agriculture's sizable effect on food security and climate change, a dearth of reasonable alternatives to animal protein has led to an uncomfortable silence from policymakers. Animals cannot be optimized for food production to the extent necessary to resolve the efficiency crisis at play, and the concept of reducing animal product consumption has been met with fierce resistance from producers and consumers alike for decades. However, with recent progress in food technology, a promising solution has emerged. Alternative proteins are foods that create the experience of eating animal products without the inefficiencies and other harms involved in cycling crops through animals (Purvis and Friedrich, Foreign Policy, Fall 2022, ISSN: 0015-7228, which is incorporated by reference herein).

Alternative proteins have three key advantages over traditional animal protein sources. First, these products are far more resource-efficient than animal foods. Second, the supply chains for alternative proteins are simpler and less vulnerable to disruption than those of animal products. Third, alternative proteins are an attractive route to circumvent allergenicity concerns around traditional food proteins, including those from animal and non-animal sources. Fourth and finally, alternative proteins produce a fraction of the greenhouse gas emissions associated with the production of traditional animal protein, helping to mitigate the effects of climate change at the same time as they help the food system weather them.

Right now, the primary obstacles to wider uptake of alternative proteins are that they cost too much and don't taste as good as conventional meat or foods derived from animal proteins.

Accordingly, there is a need for new methods of predicting, evaluating, and/or producing alternative proteins with target food functions, which may be related to their physicochemical properties.

### SUMMARY

Provided herein is a method of predicting at least one target food function of a candidate protein, the method comprising providing an amino acid sequence for the candidate protein; computer processing, by at least one processor executing a trained computer model, the amino acid sequence of the candidate protein to predict a set of candidate amino acid sequences having intrinsic disorder, wherein the intrinsic disorder may comprise a lack of stable secondary structure along at least about 10% of the length of an individual candidate amino acid sequence; and computer processing, by at least one processor executing a trained computer model, the set of candidate amino acid sequences to generate an output that is indicative of the predicted target food function or target food functions of the candidate protein.

The method may further comprise computer processing the set of candidate amino acid sequences to determine a set of physicochemical properties of individual candidate amino acid sequences.

The method may further comprise computer processing of the set of physicochemical properties to generate the output.

The set of physicochemical properties may comprise one or more of: sequence length, molecular weight, isoelectric point, pH-adjusted net charge per residue, a presence or number of hydrophobic amino acids within the sequence, a presence or number of aliphatic amino acids within the sequence, a presence or number of aromatic amino acids within the sequence, a presence or number of positively charged amino acids within the sequence, a presence or number of negatively charged amino acids within the sequence, a presence or number of polar amino acids within the sequence, a presence or number of glycine amino acids within the sequence, a presence or number of alanine amino acids within the sequence, a presence or number of cysteine amino acids within the sequence, a presence or number of proline amino acids within the sequence, a presence or number of histidine amino acids within the sequence, a distribution of hydrophobic amino acids within the sequence, a distribution of aliphatic amino acids within the sequence, a distribution of aromatic amino acids within the sequence, a distribution of positively charged amino acids within the sequence, a distribution of negatively charged amino acids within the sequence, a distribution of polar amino acids within the sequence, a distribution of glycine amino acids within the sequence, a distribution of alanine amino acids within the sequence, a distribution of cysteine amino acids within the sequence, a distribution of proline amino acids within the sequence, and a distribution of histidine amino acids within the sequence; wherein the distribution may be expressed in terms of the mean inverse distance weight parameter of amino acids within the sequence.

A method of predicting a target food function of a candidate protein, the method comprising: providing an amino acid sequence for the candidate protein; computer processing the amino acid sequence of the candidate protein to generate a set of candidate amino acid sequences and a set of physicochemical properties of individual candidate amino acid sequences, wherein the set of physicochemical properties may comprise one or more of: sequence length, molecular weight, isoelectric point, pH-adjusted net charge per residue, a presence or number of hydrophobic amino acids within the sequence, a presence or number of aliphatic amino acids within the sequence, a presence or number of aromatic amino acids within the sequence, a presence or number of positively charged amino acids within the sequence, a presence or number of negatively charged amino acids within the sequence, a presence or number of polar amino acids within the sequence, a presence or number of glycine amino acids within the sequence, a presence or number of alanine amino acids within the sequence, a presence or number of cysteine amino acids within the sequence, a presence or number of proline amino acids within the sequence, a presence or number of histidine amino acids within the sequence, a distribution of hydrophobic amino acids within the sequence, a distribution of aliphatic amino acids within the sequence, a distribution of aromatic amino acids within the sequence, a distribution of positively charged amino acids within the sequence, a distribution of negatively charged amino acids within the sequence, a distribution of polar amino acids within the sequence, a distribution of glycine amino acids within the sequence, a distribution of alanine amino acids within the sequence, a distribution of cysteine amino acids within the sequence, a distribution of proline amino acids within the sequence, and a distribution of histidine amino acids within the sequence, wherein the distribution may be expressed in terms of the mean inverse distance weight parameter of amino acids within the sequence; and computer processing the set of candidate amino acid sequences and the set of physicochemical properties to generate an output that is indicative of whether the candidate protein has the target food function.

The trained computer model may be trained using a training data set comprising: a set of amino acid sequences of individual proteins with the target food function; and/or a set of amino acid sequences of individual proteins without the target food function.

The trained computer model may be trained using a training data set comprising: a set of amino acid embeddings of individual proteins with the target food function derived from a protein large language model (pLLM); and/or a set of amino acid sequences of individual proteins without the target food function derived from a pLLM.

The trained computer model may be trained using a training data set comprising one of a set of physicochemical properties of a set of amino acid sequences of individual proteins with the target food function, and a set of physicochemical properties of a set of amino acid sequences of individual proteins without the target food function.

The trained computer model may comprise a trained machine learning model.

The machine learning model may comprise one or more of a classifier model, a regression model, a distance model, or a neural network model.

The set of physicochemical properties may comprise a presence or number of clustering motifs within the sequence.

The set of physicochemical properties may comprise: a distribution of clustering motifs within the sequence, wherein the distribution is expressed in terms of the mean inverse distance weight parameter of clustering motifs within the sequence.

The set of physicochemical properties may comprise one or more normalized sequence asymmetry parameters between two different types of individual amino acids or two different groups of amino acids.

The method may further comprise recombinantly expressing one or more candidate amino acid sequences within the set of candidate amino acid sequences to provide quantities of candidate proteins.

The method may further comprise conducting analytical assays to validate the target food function for each of the candidate proteins.

The method may further comprise further training or fine-tuning the trained computer model using data obtained from the analytical assays.

The method may further comprise selecting one or more of the candidate proteins as potential food ingredients if the individual candidate proteins are determined to have the target food function satisfying a predetermined criterion.

The method may further comprise assessing the one or more candidate proteins selected as potential food ingredients to determine whether the candidate proteins meet desired performance requirements as part of a food preparation.

The candidate protein may be naturally occurring in one of plants, fungi, protists, and insects.

The candidate protein may be synthetic.

The candidate protein may be a domain of a larger protein.

The target food function may comprise phase separation of the candidate protein or a domain thereof from an aqueous solution upon exposure to one or more environmental triggers to form a dense phase and a light phase, wherein the target food function is inversely proportional to the concentration of the candidate protein or a domain thereof in the light phase.

The environmental triggers may comprise pH of the aqueous solution.

The one or more environmental triggers may comprise a salt concentration greater than 0 mM in the aqueous solution.

The one or more environmental triggers may comprise the temperature of the aqueous solution.

The one or more environmental triggers may comprise a food additive concentration greater than 0 mM in the aqueous solution.

The one or more environmental triggers may comprise one or more enzymes.

The one or more enzymes may comprise a protease enzyme.

The set of physicochemical properties may comprise a presence or number of enzyme cleavage sites within the sequence.

The cleavage of the candidate protein by one or more enzymes may provide a C-terminal domain of the candidate protein, wherein the C-terminal domain has the target food function.

The cleavage of the candidate protein by one or more enzymes may provide a N-terminal domain of the candidate protein, wherein the N-terminal domain has the target food function.

The cleavage of the candidate protein by one or more enzymes may provide a central domain of the candidate protein, wherein the central domain has the target food function.

The cleavage of the candidate protein by one or more enzymes may provide two or more domains of the candidate protein, wherein the two or more domains have the target food function.

At least one analytical assay may determine the concentration of the candidate protein in aqueous solution as a variable function of one or more environmental conditions.

The concentration of the candidate protein in aqueous solution may be determined by at least measuring the concentration of the candidate protein in the light phase.

The one or more environmental conditions may comprise pH, salt concentration, temperature, additive concentrations, and/or enzyme presence or concentration.

The predetermined criterion may comprise the target food function under one or more sets of one or more environmental conditions.

The one or more candidate proteins selected as potential food ingredients have a concentration in aqueous solution below a predetermined threshold under a predetermined set of environmental conditions.

A composition may further comprise a candidate protein predicted to have the target food function according to the above methods, and one or more food ingredients.

A food product may further comprise a candidate protein predicted to have the target food function according to the above methods.

A composition may further comprise: an individual protein, wherein the individual protein may comprise a primary amino acid sequence that is conserved by 50% or more relative to any one of SEQ ID NOs: 1 to 43; and one or more food ingredients.

A food product may further comprise the composition of any of the above compositions.

Other aspects and features will become apparent to those ordinarily skilled in the art, upon review of the following description of some exemplary embodiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The content of the electronically submitted Sequence Listing (name: “P1321US_Sequence listing.xml”; size: 56.036 bytes; and date of creation: Dec. 22, 2023), is herein incorporated by reference in its entirety.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings. The drawings included herewith are for illustrating various examples of articles, methods, and apparatuses of the present specification. In the drawings:

FIG. 1 shows a graphic overview of a discovery platform.

FIG. 2 shows additional details of how the discovery platform of FIG. 1 computes a disordered protein database by mining protein sequence repositories.

FIG. 3 shows a graphical depiction of protein disorder as found in intrinsically disordered proteins (IDPs) and proteins comprising intrinsically disordered regions (IDRs).

FIG. 4 shows additional details of how the discovery platform of FIG. 1 comprises decoding of a target functionality to associate the defined target functionality (e.g., a target food function) with target sequences and user-defined properties (e.g., physicochemical properties).

FIG. 5 shows how a protein sequence decoder can extract functionality (such as in identifying ‘hit’ proteins with the desired target functionality 600) from a target sequence.

FIG. 6 shows how a material property decoder can extract functionality (such as in identifying ‘hit’ proteins with a desired target functionality) based on user-defined properties by using machine learning models (MLMs) or feature-based models (FBMs) or a combination of both to identify hits in a disordered protein database.

FIG. 7 shows additional details of how the discovery platform of FIG. 1 comprises a computational validation pipeline to further evaluate and filter preliminary hit sequences.

FIG. 8 shows additional details of how the discovery platform of FIG. 1 comprises experimental validation (e.g., analytical assays) to identify experimentally validated hits among the computationally validated hits.

FIG. 9 shows examples of how validated protein ingredients predicted and validated by the discovery platform of FIG. 1 can be used to craft consumer food products.

FIG. 10 shows a schematic illustration of a target food function, wherein the target food function is phase separation of the protein from an aqueous solution to form a light phase and a dense phase following exposure to one or more environmental triggers.

FIG. 11 shows a schematic illustration of a target food function, wherein the target food function is phase separation of the protein from an aqueous solution to form a light phase and a dense phase following exposure to one or more environmental triggers, wherein the environmental triggers comprise a protease enzyme.

FIG. 12 shows illustrative examples of qualitative (A) and quantitative (B) phase separation assays to validate the desired target functionality in a candidate protein.

FIG. 13 shows a photograph of a candidate protein gel (A) and illustrative examples of rheology measurements (B).

FIG. 14 shows photographs of cheese curds prepared according to the compositions disclosed in Example 6, with either a single candidate protein (A) or a candidate protein blend (B).

### DETAILED DESCRIPTION

In the following detailed description, reference is made to the accompanying figures, which form a part hereof. In the figures, similar symbols may identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, and personal computer, cloud-based program or system, laptop, personal data assistance, cellular telephone, smartphone, or tablet device.

Each program is preferably implemented in a high-level procedural or object-oriented programming and/or scripting language to communicate with a computer system. However, the programs can be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein.

A description of an embodiment with several components in communication with each other does not imply that all such components are required. On the contrary, a variety of optional components are described to illustrate the wide variety of possible embodiments of the present invention.

Further, although process steps, method steps, algorithms or the like may be described (in the disclosure and/or in the claims) in a sequential order, such processes, methods, and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

### Definitions

Throughout this application, various embodiments may be presented in a range of formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including combinations thereof.

The terms “determining,” “measuring,” “evaluating,” “assessing,” “assaying,” and “analyzing” are often used interchangeably herein to refer to forms of measurement. The terms include determining whether an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values, can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

The term “protein” (e.g., an individual protein, a candidate protein, a traditional food protein, an alternative protein, etc.) inherently comprises a primary amino acid sequence that is characteristic to the protein such that a reference to the primary amino acid sequence is also a reference to the protein and vice versa.

The term “hydrophobic amino acids” as used herein generally refers to amino acids comprising one or more of: valine (Val, V), leucine (Leu, L), isoleucine (Ile, I), methionine (Met, M), phenylalanine (Phe, F), tyrosine (Tyr, Y), and tryptophan (Trp, W).

The term “aliphatic amino acids” as used herein generally refers to amino acids comprising one or more of: valine (Val, V), leucine (Leu, L), isoleucine (Ile, I), and methionine (Met, M).

The term “aromatic amino acids” as used herein generally refers to amino acids comprising one or more of: phenylalanine (Phe, F), tyrosine (Tyr, Y), and tryptophan (Trp, W).

The term “positively charged amino acids” as used herein generally refers to amino acids comprising one or more of: arginine (Arg, R), and lysine (Lys, K).

The term “negatively charged amino acids” as used herein generally refers to amino acids comprising one or more of: aspartic acid (Asp, D), and glutamic acid (Glu, E).

For the purposes of the disclosure, it shall be understood that “polar amino acids” comprise one or more of: asparagine (Asn, N), glutamine (Gln, Q), serine (Ser, S), and threonine (Thr, T).

Other amino acids relevant to the disclosure in addition to those listed above include: alanine (Ala, A), cysteine (Cys, C), glycine (Gly, G), histidine (His, H), proline (Pro, P), selenocysteine (Sec, U), 4-hydroxyproline (Hyp, O), and pyroglutamic acid (Glp, U).

### THE TECHNOLOGY

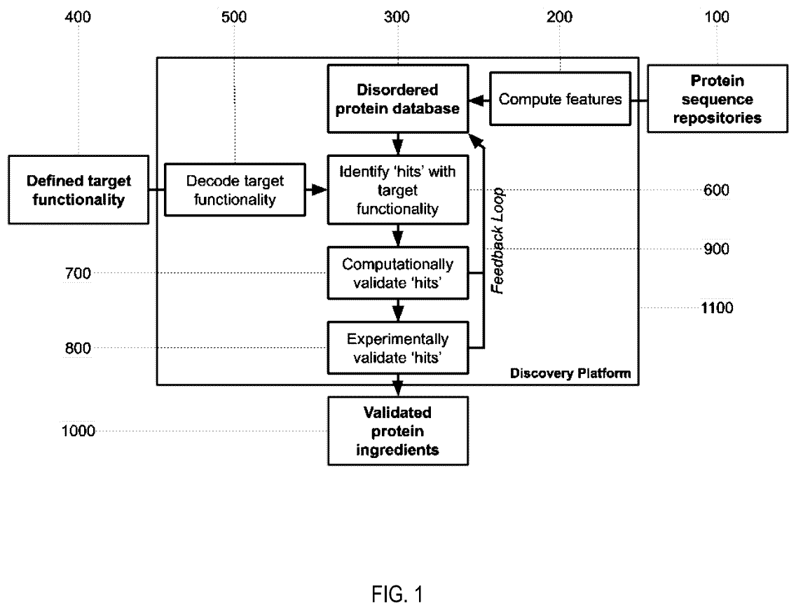

Referring now to FIG. 1, therein is shown a graphic overview of a discovery platform 1100. The discovery platform 1100 is configured to predict and validate a defined target functionality (e.g., a target food function) 400 in a candidate protein selected from a disordered protein database 300 to provide validated protein ingredients (e.g., candidate proteins with the target food function) 1000. Some of the steps of FIG. 1 are further discussed below with reference to other figures.

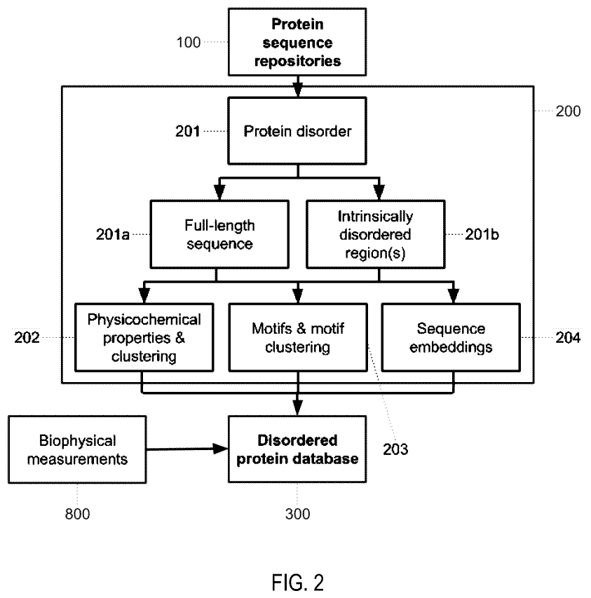

FIG. 2 shows additional details of how the discovery platform 1100 computes the disordered protein database 300 by mining protein sequence repositories 100, wherein the mining comprises computer processing (“compute features”) 200 to identify hidden higher order sequence features relating to protein functions. The first step of the computer processing 200 is to calculate protein disorder along the full-length input sequence 201a to yield all intrinsically disordered regions (IDRs) 201b. Subsequent computer processing 200 of sequences 201a (representing full-length sequences) and 201b (representing intrinsically disordered regions) calculates the physicochemical properties 202 of the full-length sequence 201a or any disordered subsequence thereof 201b; the occurrence, asymmetry and clustering of sequence features 203 in 201a and 201b (e.g., intermolecular and/or intramolecular interactions associated with one or more clustering motifs); and a numerical embedding of the sequences 201a and 201b using a pre-trained and fine-tuned protein language model 204. In addition, any experimental measurements (e.g., “biophysical measurements”) 800 of the macroscale biophysical properties of proteins 201a and 201b generated by the discovery platform 1100 of FIG. 1 are included in the disordered protein database 300 to establish feedback loops 900 as shown in FIG. 1.

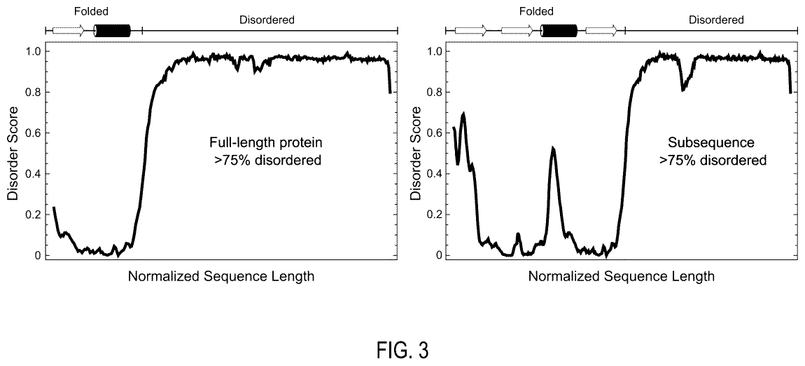

FIG. 3 shows a graphical depiction of protein disorder as found in intrinsically disordered proteins (IDPs) and proteins comprising intrinsically disordered regions (IDRs). The graphical depiction shows how some regions of an IDP or proteins comprising an IDR can form stable secondary structures while other regions of said proteins can be disordered wherein no stable secondary structure forms. The graphical depiction includes an illustration of how disorder in a protein can be predicted based upon a disorder score that can vary along the length of the primary amino acid sequence of the protein. The disorder score can be obtained by computer processing the primary amino acid sequence.

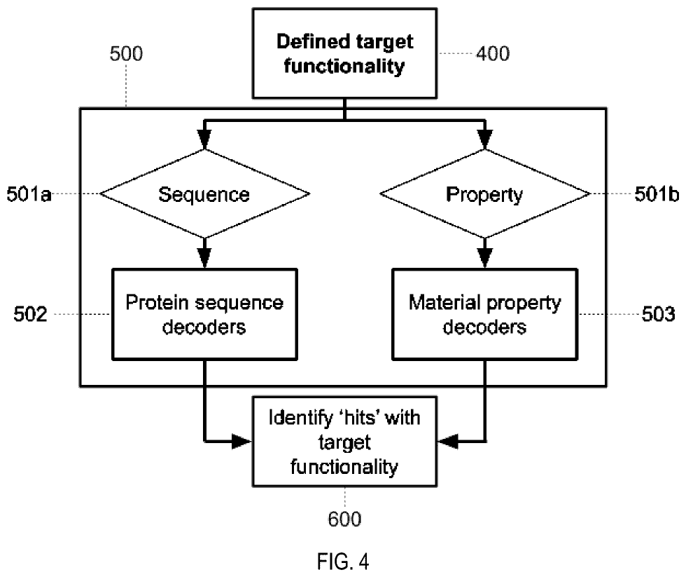

FIG. 4 shows additional details of how the discovery platform 1100 comprises decoding of the target functionality 500 to associate the defined target functionality (e.g., the target food function) 400 with target sequences 501a and user-defined properties (e.g., physicochemical properties) 501b. Identifying target sequences 501a and user-defined properties 501b in proteins within the disordered protein database 300 can provide ‘hit’ proteins with the desired target functionality 600. Decoding of the target functionality 500 can be achieved using computer processing. Said computer processing can comprise using protein sequence decoders 502 and/or material property decoders 503.

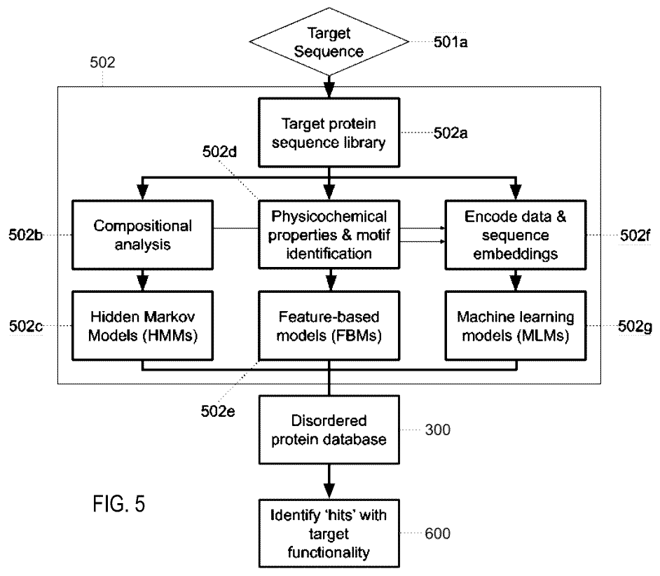

FIG. 5 shows how a protein sequence decoder 502 can extract functionality (such as in identifying ‘hit’ proteins with the desired target functionality 600) from a target sequence 501a. A protein sequence library 502a can be compiled by collecting and curating sequence homologs from public and in-house databases, or experimentally by compositional or saturation mutagenesis of the target sequence 501a. The sequence composition 502b of the sequence library 502a can then be analyzed and encoded into Hidden Markov Models (HMMs) 502c. Physicochemical properties and motifs 502d within the target sequence 501a are analyzed and used to encode functionality in feature-based models (FBMs) 502e. Sequences can also be directly embedded or enriched with information about sequence composition, physicochemical properties and motifs 502f and used to train machine learning models (MLMs) 502g. The models can be used individually or in combination to identify hits with desired functionality 600 in the disordered protein database 300.

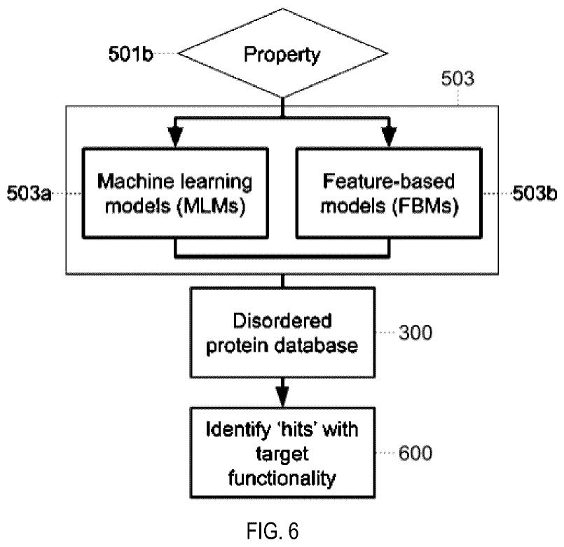

FIG. 6 shows how a material property decoder 503 can extract functionality (such as in identifying ‘hit’ proteins with the desired target functionality 600) based on user-defined properties 501b by using machine learning models (MLMs) 503a or feature-based models (FBMs) 503b or a combination of both to identify hits 600 in a disordered protein database 300.

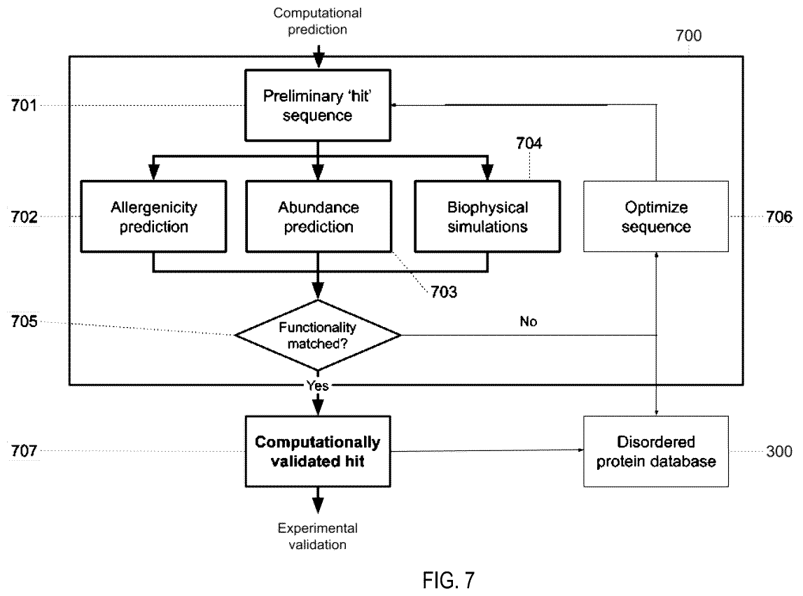

FIG. 7 shows additional details of how the discovery platform 1100 comprises a computational validation pipeline 700 to further evaluate and filter preliminary hit sequences 701. Allergenicity predictions 702 are used to filter out preliminary hits 701 at risk of triggering allergic reactions. Abundance predictions 703, based on measurements, are used to filter out preliminary hits 701 that are not sufficiently abundant in target species. Biophysical simulations 704 are used to validate the presence of the desired target functionality 400 in ‘hit’ proteins 600. The simulation 704 can comprise computer processing the primary amino acid sequence 701 of a ‘hit’ protein with the desired target functionality 600. The computer processing can comprise coarse-grained simulations and/or all-atom simulations. The modules 702, 703 and 704 can either be used individually or in combination. If the preliminary hit sequence 701 passes at least the allergenicity prediction filter 702 (Yes in 705), the computationally validated hit 706 can proceed for experimental validation 800. If the test 705 fails (No in 705), the protein sequence is optimized by rational or random sequence changes 706, and the pipeline 700 is conducted again in an iterative process. All data obtained from the pipeline 700 can be deposited into the disordered protein database 300 to improve the discovery platform 1100 as part of the feedback loop 900.

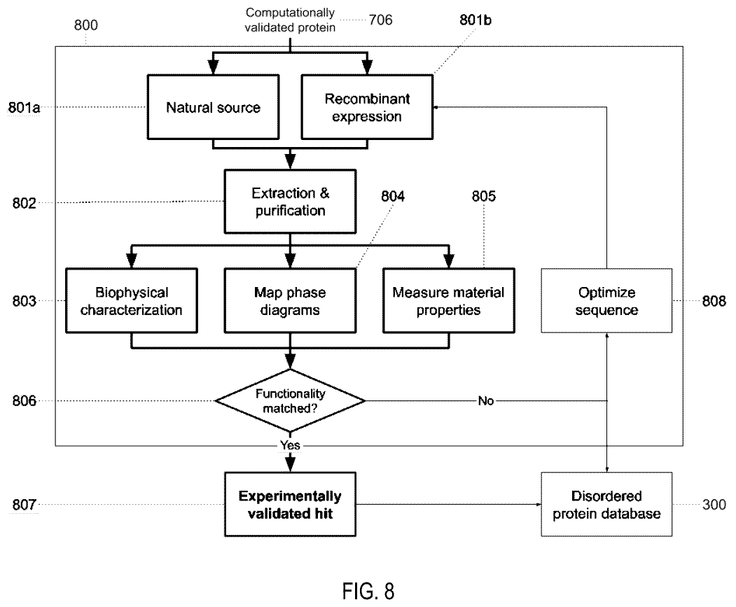

FIG. 8 shows additional details of how the discovery platform 1100 comprises experimental validation (e.g., analytical assays) 800 to identify experimentally validated hits 807 among the computationally validated hits 706. Quantities of computationally validated hits 706 can be obtained by extraction and purification 802 from either natural sources 801a or via recombinant expression 801b. Said quantities can subsequently be subjected to analytical assays that comprise one or more of biophysical characterization 803, mapping of phase diagrams 804, and measuring of material properties 805. If, based upon the results of the analytical assays, the target functionality 400 is matched (Yes in 806), the experimentally validated hit 807 can be classified as a validated protein ingredient 1000 and proceed for evaluation for incorporation into commercial food products. If, based upon the results of the analytical assays, the target functionality 400 is not matched (No in 806), the protein sequence is optimized by rational or random sequence changes 808, and the experimental validation 800 is conducted again in an iterative process. All data from the experimental validation 800 can be deposited into the disordered protein database 300 to improve the discovery platform 1100.

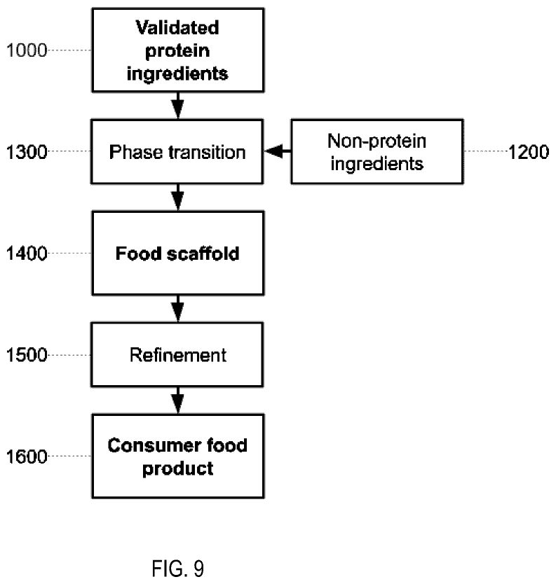

FIG. 9 shows examples of how validated protein ingredients 1000 predicted and validated by the discovery platform 1100 can be used to craft consumer food products 1600. Validated protein ingredients 1000 can be combined with non-protein food ingredients 1200 and triggered to undergo a phase transition 1300 to yield a food scaffold 1400. The scaffold 1400 can be subsequently refined (e.g., processed, optimized, and/or combined with other food ingredients) to provide a consumer food product 1600. FIGS. 9, 10, 11, and 12 discuss phase transition/separation as the target food function, however, food functions other than phase transition/separation may be the target food function.

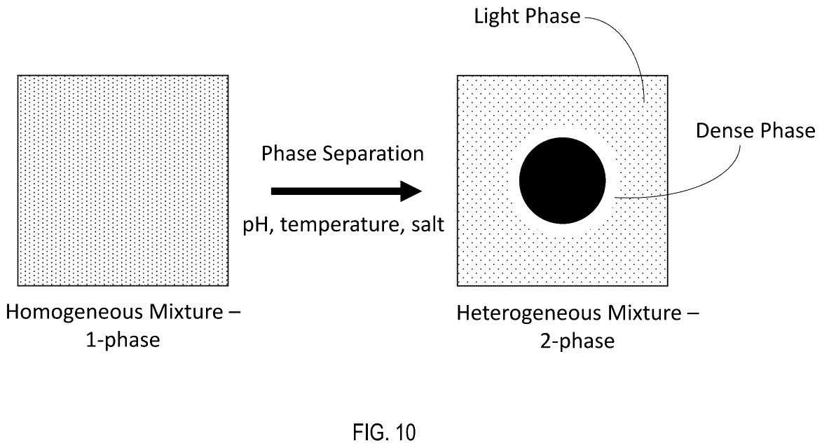

FIG. 10 shows a schematic illustration of a target food function, wherein the target food function is phase separation of the protein from an aqueous solution to form a light phase and a dense phase following exposure to one or more environmental triggers. In some embodiments the one or more environment triggers may include pH, temperature, or salt concentration. In other embodiments other environmental triggers may be used to achieve the target food function.

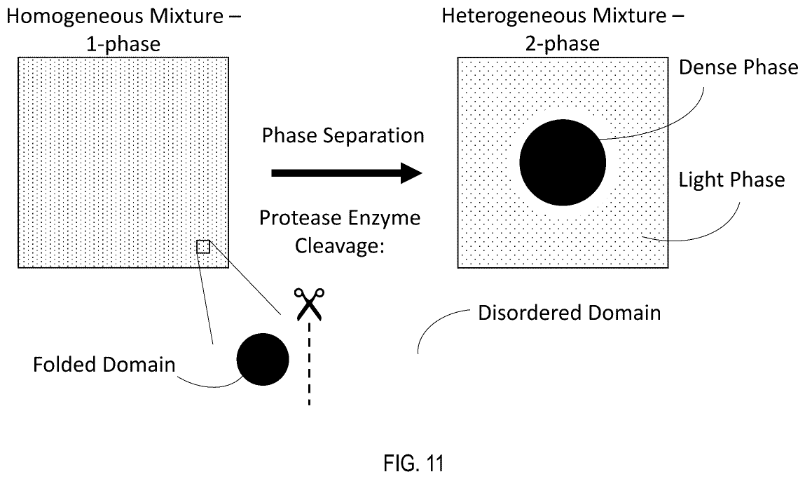

FIG. 11 shows a schematic illustration of a target food function, wherein the target food function is phase separation of the protein from an aqueous solution to form a light phase and a dense phase following exposure to one or more environmental triggers, wherein, in the embodiment of FIG. 11, the environmental triggers for phase separation comprise a protease enzyme. In some embodiments where a protease enzyme is an environmental trigger there may be additional environmental triggers required for optimal functioning of the protease, for example pH.

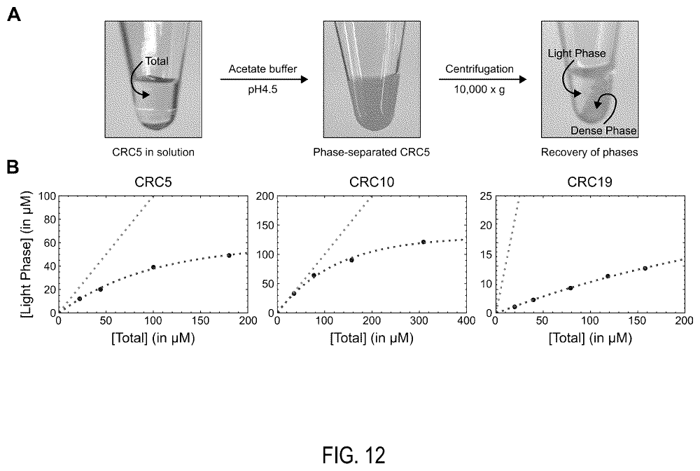

FIG. 12 shows illustrative examples of qualitative (A) and quantitative (B) phase separation assays to validate the desired target functionality in a candidate protein. In FIG. 12A, lowering pH is the environmental trigger for phase separation of protein CRC5. The resulting light and dense phases are recovered by centrifugation. In FIG. 12B, the same assay is performed for three proteins CRC5, CRC10, and CRC19 at various starting concentrations, denoted [Total], and plotted as the x axis. After pH-induced phase separation and recovery of the light and dense phases by centrifugation, the remaining protein concentration in the light phases is measured, denoted [Light Phase], and plotted on the y axis.

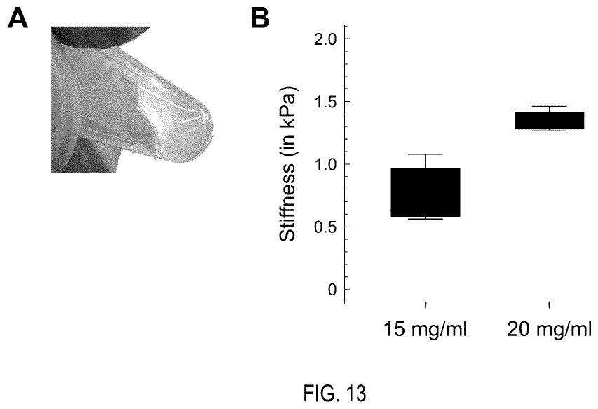

FIG. 13 shows a photograph of a candidate protein gel (A) and illustrative examples of rheology measurements to quantify and compare the stiffness of CRC gels formed at various concentrations (B). In the embodiment of FIG. 13, CRC5 gels are formed via a pH-induced liquid-to-solid phase transition with protein CRC5 at 15 mg/ml and 20 mg/ml starting (i.e., light phase) concentrations. In some embodiments, the light phase concentrations and environmental triggers may be alternative or additional environmental triggers depending on the target protein and desired emergent material properties.

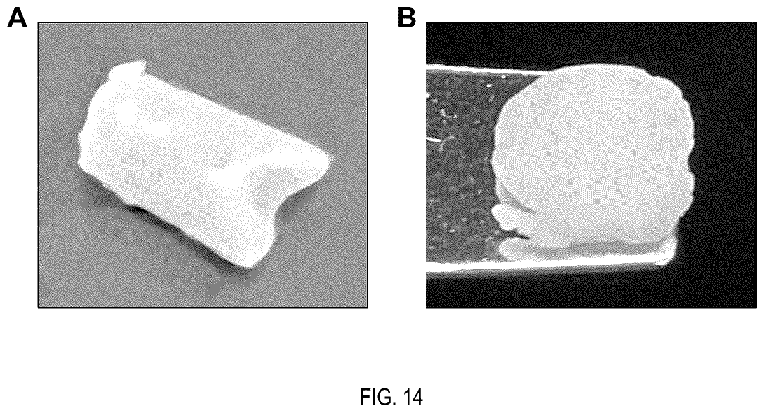

FIG. 14 shows photographs of cheese curds prepared according to the compositions disclosed in Example 6, with either a single candidate protein (A) or a candidate protein blend (B).

### Advantages of the Technology

In an aspect, disclosed herein are methods of predicting a target food function of a candidate protein. In some embodiments, the candidate protein is an individual protein. In an aspect, provided herein are compositions comprising individual proteins and one or more food ingredients. In some embodiments, the candidate protein or the individual protein is used as a food ingredient. In some embodiments, a food product comprises the candidate protein or the individual protein.

The methods disclosed herein encompass a discovery platform built around specialized knowledge of intrinsically disordered proteins (IDPs) and proteins comprising intrinsically disordered regions (IDRs) that can enable the prediction, evaluation, and/or validation of a target food function in a candidate protein. The candidate protein may find subsequent use as a food ingredient or as an alternative protein in food. Previously, IDPs and IDRs have almost exclusively been studied in the context of their cellular functions, with most insights dating back less than 10 years (Holehouse and Kragelund, Nat. Rev. Mol. Cell Biol. 2023, which is incorporated as a reference herein).

Yet, it turns out that many traditional food proteins, such as casein (e.g., bovine casein), are IDPs or comprise an IDR. Further, these traditional food proteins that are IDPs or comprise an IDR have one or more target food functions that a replacement protein (e.g., an alternative protein) must also have, at least one of said target food functions to a predetermined degree or threshold. The lack of available methods for the prediction, evaluation, and/or validation of a target food function in IDPs or proteins that comprise an IDR has historically limited the systematic identification and development of candidate proteins that may serve as alternative proteins to replace traditional food proteins that are IDPs or comprise an IDR. A candidate protein may be an IDPs or comprise an IDR. A candidate protein may not be an IDPs or comprise an IDR while still having a target food function found in a traditional food protein that is an IDPs or comprises an IDR.

The methods disclosed herein may allow a practitioner to identify the features and/or physicochemical properties of a candidate protein (e.g., the primary amino acid sequence of the candidate protein) that are associated with the target food function. Practice of the method may allow the practitioner to identify relationships between the primary amino acid sequence, physicochemical properties, and target food functions of the candidate protein. Practice of the methods disclosed herein comprises computer processing of at least the amino acid sequence of the candidate protein. Output obtained by the methods disclosed herein can indicate the presence or absence of a target food function in a candidate protein.

Evaluation or validation of a target food function in a candidate protein can comprise analytical assays. In some embodiments, analytical assays comprise measurement or validation of the physicochemical properties and/or target food function of the candidate protein. Data obtained from analytical assays can be utilized to improve the methods disclosed herein.

### Alternative Proteins

In an aspect, disclosed herein are methods of predicting a target food function of a candidate protein. In some embodiments, the candidate protein is an individual protein. In some embodiments, the individual protein is a candidate protein. In some embodiments, the candidate protein occurs in nature. In some embodiments, the candidate protein does not occur in nature. In some embodiments, the method is practiced to at least determine the suitability of a candidate protein to serve as an alternative protein. In some embodiments, the individual protein is used as an alternative protein.

Alternative proteins may have one or more target food functions that allow them to be incorporated into food products as a replacement for one or more traditional food proteins. Traditional food proteins may be derived from an animal or plant source that is obtained according to modern agricultural methods. For example, proteins derived from peas have been used as alternative proteins for traditional food proteins present in beef, wherein the alternative proteins are incorporated into a food product intended to replicate the properties of beef. Use of alternative proteins in place of traditional food proteins can reduce the greenhouse gas emissions associated with the production of food. An alternative protein may comprise the same or similar physicochemical and/or material properties as a traditional food protein it emulates in a food product without triggering allergic reactions in consumers of the alternative protein who may experience allergic reactions following consumption of the traditional food protein. In some embodiments, a candidate protein is an alternative protein. In some embodiments, an individual protein is an alternative protein. Traditional food proteins may be found and/or derived from animals, plants, fungi, protists, or other sources of food or food ingredients.

### Intrinsically Disordered Proteins

Many proteins adopt characteristic three-dimensional structures comprising secondary, tertiary, and quaternary structures that may be readily predicted on the basis of the primary amino acid sequences of said proteins. Secondary structure of a protein may comprise alpha helices and/or beta pleated sheets. The formation of secondary structure by a protein (e.g., a primary amino acid sequence) may be a prerequisite for accurately predicting the three-dimensional structure of said protein. The potential for a protein to form one or more stable secondary structures may be readily discerned by analysis of the primary amino acid sequence, as shown, and discussed above for FIG. 3. The presence of one or more stable secondary structures in a protein may be readily determined by analytical assays.

Proteins and/or domains thereof that do not form stable secondary structures may be referred to as intrinsically disordered proteins (IDPs) and/or intrinsically disordered regions (IDRs). The lack of stable secondary structure in IDPs and IDRs may preclude the formation of stable three-dimensional structures by IDRs and IDPs. While accurate three-dimensional structural data (e.g., as in a crystal structure) is available for large databases of ordered proteins with stable secondary structure, there is a relative scarcity of three-dimensional structural data for IDPs and IDRs. IDPs and proteins comprising IDRs may display no stable three-dimensional structure, such that existing methods of prediction optimized for ordered proteins may be unsuitable for adaptation for IDPs and proteins comprising IDRs. Predicting the shape, physicochemical properties, and/or target food functions of an IDP or IDR may be much more challenging in comparison to predicting similar features of ordered proteins that form stable secondary and tertiary structures and/or have characteristic three-dimensional structures.

In some embodiments, a candidate protein is an IDP. In some embodiments, a candidate protein comprises an IDR. An IDP may be alternatively termed a protein with intrinsic disorder. When a protein contains one or more IDRs, said one or more IDRs may be referred to as one or more domains of a protein with intrinsic disorder. Due to their lack of stable secondary structure, proteins, and domains thereof with intrinsic disorder may not form a stable three-dimensional structure in isolation and/or upon binding to a partner, although structure may be acquired upon binding.

Secondary structure refers to regular, recurring arrangements in space of adjacent amino acid residues in a polypeptide chain (e.g., a primary amino acid sequence). Secondary structure in a protein may comprise intramolecular interactions. The formation of alpha helices and/or beta pleated sheets may be predictable based upon the physicochemical properties associated with the primary amino acid sequence of a protein, wherein certain amino acids may form intermolecular interactions with other amino acids within the same protein in a regular and recurrent fashion. The lack of stable secondary structure in IDPs and IDRs suggests that alternative features and physicochemical properties may dictate their intramolecular interactions, intermolecular interactions, and/or target food functions. The features and physicochemical properties of IDPs and IDRs that dictate their intramolecular interactions, intermolecular interactions, and/or target food functions may be distinct from the features and physicochemical properties of ordered proteins that dictate their intramolecular interactions, intermolecular interactions, and/or target food functions.

An IDP may comprise one or more IDRs in addition to one or more regions that independently comprise a stable secondary structure. In some embodiments, an IDP comprises a dynamic (e.g., fluxional) three-dimensional structure owing to the presence of one or more IDRs in said IDP.

In some embodiments, the methods disclosed herein may provide correlations between the physicochemical properties of candidate proteins and the intramolecular interactions they form. In some embodiments, the methods disclosed herein may provide correlations between the physicochemical properties of candidate proteins and the intermolecular interactions they form. In some embodiments, the methods disclosed herein may provide correlations between the physicochemical properties of candidate proteins and the degree of a target food function in said candidate proteins.

In some embodiments, the presence of intrinsic disorder in a candidate protein or a domain thereof is predicted on the basis of the primary amino acid sequence of the candidate protein or the domain thereof. In some embodiments, the presence of intrinsic disorder in a candidate protein or a domain thereof is predicted by computer processing the primary amino acid sequence of the candidate protein or the domain thereof.

In some embodiments, the candidate protein has intrinsic disorder. In some embodiments, the candidate protein comprises at least one intrinsically disordered region. A candidate protein that has intrinsic disorder may include a candidate protein comprising at least one intrinsically disordered region. In some embodiments, a protein with a domain that has intrinsic disorder is a protein with at least one intrinsically disordered region. In some embodiments, the candidate protein is determined to have intrinsic disorder by computer processing the primary amino acid sequence of the candidate protein. In some embodiments, the candidate protein is determined to not have intrinsic disorder by computer processing the primary amino acid sequence of the candidate protein.

In some embodiments, a candidate protein is determined to have intrinsic disorder when there is a lack of stable secondary structure along at least about 10% to about 100% of the length of the candidate protein. In some embodiments, a candidate protein is determined to have intrinsic disorder when there is a lack of stable secondary structure along at least about 30% to about 100% of the length of the candidate protein. In some embodiments, a candidate protein is determined to have intrinsic disorder when there is a lack of stable secondary structure along at least about 50% to about 100% of the length of the candidate protein. In some embodiments, a candidate protein is determined to have intrinsic disorder when there is a lack of stable secondary structure along at least about 75% to about 100% of the length of the candidate protein. In some embodiments, a candidate protein is determined to have intrinsic disorder when there is a lack of stable secondary structure along at least about 10% of the length of the candidate protein. In some embodiments, a candidate protein is determined to have intrinsic disorder when there is a lack of stable secondary structure along at least about 20% of the length of the candidate protein. In some embodiments, a candidate protein is determined to have intrinsic disorder when there is a lack of stable secondary structure along at least about 30% of the length of the candidate protein. In some embodiments, a candidate protein is determined to have intrinsic disorder when there is a lack of stable secondary structure along at least about 40% of the length of the candidate protein. In some embodiments, a candidate protein is determined to have intrinsic disorder when there is a lack of stable secondary structure along at least about 50% of the length of the candidate protein. In some embodiments, a candidate protein is determined to have intrinsic disorder when there is a lack of stable secondary structure along at least about 60% of the length of the candidate protein. In some embodiments, a candidate protein is determined to have intrinsic disorder when there is a lack of stable secondary structure along at least about 70% of the length of the candidate protein. In some embodiments, a candidate protein is determined to have intrinsic disorder when there is a lack of stable secondary structure along at least about 80% of the length of the candidate protein. In some embodiments, a candidate protein is determined to have intrinsic disorder when there is a lack of stable secondary structure along at least about 90% of the length of the candidate protein. In some embodiments, a candidate protein is determined to have intrinsic disorder when there is a lack of stable secondary structure along at least about 100% of the length of the candidate protein.

### Target Food Functions

Candidate proteins useful as alternative proteins for food products may have one or more target food functions. A target food function may comprise a mechanical, chemical, physicochemical, and/or sensory property of the candidate protein. A target food function may be advantageous to the purification or isolation of the candidate protein. A target food function may be advantageous to the processing of the candidate protein for use in a food product. A target food function may be advantageous to the production of the candidate protein. A target food function may comprise a sensory property of the protein that impacts the impression of a consumer (e.g., a human subject) of a food product comprising the candidate protein.

The subject matter of this disclosure can be used to identify individual proteins with target food functions that are desirable for incorporation into food products. In some embodiments, the individual proteins with target food functions are alternative proteins that may serve as replacements for traditional food proteins that have at least one of said target food functions. The individual proteins with target food functions may be desirable as food ingredients because, as non-limiting examples: they are obtainable from a more sustainable and environmentally friendly form of agriculture or harvesting, they are less expensive to produce, they are non-allergenic, and/or they have other beneficial characteristics relevant to at least one of their sourcing, production, processing, manipulation, alteration (e.g., physical or chemical), and/or incorporation into a food product.

In some embodiments, the target food function may be dependent on one or more environmental triggers, wherein a change in an environmental parameter enables the target food function. In some embodiments, the target food function may be dependent on one or more environmental conditions that act as environmental triggers. In some embodiments, an individual protein has more than one target food function. In some embodiments, the target food function is reversible (e.g., a reversible physicochemical process). In some embodiments, the target food function is irreversible (e.g., an irreversible physicochemical process). In some embodiments, the target food function is phase separation of the protein (e.g., the individual protein) from aqueous solution. In some embodiments, the target food function is phase separation of the protein (e.g., the individual protein) from non-aqueous solution. Phase separation is discussed above for FIGS. 9, 10, 11, and 12. In some embodiments, the target food function is solubility of the individual protein in aqueous solution. In some embodiments, the target food function is solubility of the individual protein in non-aqueous solution. In some embodiments, the target food function is chemical and material stability. In some embodiments, the target food function is the formation of a gel comprising the individual protein. In some embodiments, the target food function is the formation of a stable foam produced from a solution comprising the individual protein. In some embodiments, the target food function is acting as a carrier. For example, a carrier may enable the incorporation and/or dispersion of one or more components into a food product, wherein the components may provide flavor, color, vitamins (e.g., vitamin B12, vitamin C), porphyrin, heme, carbohydrate, and/or fat. In some embodiments, the target food function is antimicrobial activity, including resistance to spoilage of a food product comprising the individual protein. In some embodiments, the target food function is nutrition (e.g., a nutritionally complete amino acid composition). In some embodiments, the target food function is viscosity modulation. In some embodiments, the target food function is moisture retention. In some embodiments, the target food function is flocculation. In some embodiments, the target food function is adhesion (e.g., adhesion of the individual protein to one or more food ingredients).

In some embodiments, the target food function is quantified, such that the degree or extent of the target food function in the candidate protein is assigned a value. In some embodiments, the target food function is quantified by analytical assays, wherein the methods of measurement, units, and metrics inherent to the analytical assays provide a basis for quantifying the target food function. One analytical assay may provide data for quantifying the target food function. More than one analytical assay may provide data for quantifying the target food function. In some embodiments, the analytical assays comprise subjective evaluation by a human subject, wherein the human subject provides data based upon their evaluation of the candidate protein. Subjective evaluation by a human subject may comprise providing said subject with a composition comprising the candidate protein for consumption as food. Further non-limiting examples of analytical assays are detailed herein.

In some embodiments, the target food function comprises phase separation of the candidate protein or a domain thereof from an aqueous solution upon exposure to one or more environmental triggers to form a dense phase and a light phase. In some embodiments, the target food function is correlated to the concentration of the candidate protein or a domain thereof in the light phase. In some embodiments, the target food function is inversely proportional to the concentration of the candidate protein or a domain thereof in the light phase. In some embodiments, the target food function is correlated to one or more properties of the dense phase (e.g., viscosity, elasticity).

Environmental triggers may comprise: pH, temperature, a salt concentration, a food additive concentration, one or more enzymes, or combinations thereof. In some embodiments, the target food function depends on a single environmental trigger. In further embodiments, the target food function depends on two or more environmental triggers.

In some embodiments, the salt comprises ions of sodium, magnesium, calcium, zinc, iron, manganese, phosphorous (e.g., phosphate), chloride, sulfate, or combinations thereof. In some embodiments, the salt comprises a chloride salt of sodium, magnesium, calcium, zinc, iron, or manganese. In some embodiments, the salt comprises a sulfate salt of sodium, magnesium, calcium, zinc, iron, or manganese. In some embodiments, the salt comprises a phosphate salt of sodium, magnesium, calcium, zinc, iron, or manganese.

In some embodiments, the food additive comprises a fat. Fats include but are not limited to: saturated and unsaturated fatty acids. Saturated and unsaturated fatty acids include, but are not limited to: oleic acid, lauric acid, palmitic acid, linoleic acid, and alpha-linoleic acid from plant and fungal sources. Plant and fungal sources include, but are not limited to: coconut milk, coconut oil, palm kernel oil, palm oil, laurel oil, pecan oil, canola oil, peanut oil, macadamia oil, sunflower oil, grape seed oil, sesame oil, sea buckthorn oil, karuka oil, nutmeg oil, soybean oil, cocoa butter, olive oil, salicornia oil, safflower oil, primrose oil, melon seed oil, artichoke oil, hemp oil, wheat germ oil, cottonseed oil, corn oil, walnut oil, rice bran oil, argan oil, pistachio oil, flax oil, hemp seed oil, lingonberry oil, and camelina oil.

In some embodiments, the food additive comprises a sugar or a fiber. Sugars and fibers include, but are not limited to: galactose, glucose, sucrose, lactose, maltose, ribose, trehalose, fructose, starch, pectins, chitins, (malto-)dextrins, and glucans from plant and fungal sources. Starches and fibers from plant and fungal sources include, but are not limited to: wheat starch, corn starch, potato starch, mung bean starch, arrowroot starch, kuzu root starch, cassava starch, tapioca starch, xanthan gum, psyllium seed husks, and chicory fiber. Glucans from plant and fungal sources include, but not limited to: agave nectar, brown rice syrup, corn syrup, coconut sugar, maple syrup, malt sugar, sugarbeet, moonrot, Selaginella, algae, shitake mushrooms, oyster mushrooms, and golden needle mushrooms.

In some embodiments, the food additive comprises a trace nutrient. Trace nutrients include, but are not limited to: L-cysteine, selenium, folate, riboflavin, vitamin E, vitamin C (ascorbic acid), vitamin A, vitamin B12, vitamin B1, vitamin B2, vitamin B3, vitamin B5, and vitamin B6.

In some embodiments, the one or more environmental triggers comprises pH. An acidic pH may be below 7. A basic pH may be above 7. In some embodiments, the pH is about 1 to about 12. In some embodiments, the pH is about 1 to about 2, about 1 to about 3, about 1 to about 4, about 1 to about 5, about 1 to about 6, about 1 to about 7, about 1 to about 8, about 1 to about 9, about 1 to about 10, about 1 to about 11, about 1 to about 12, about 2 to about 3, about 2 to about 4, about 2 to about 5, about 2 to about 6, about 2 to about 7, about 2 to about 8, about 2 to about 9, about 2 to about 10, about 2 to about 11, about 2 to about 12, about 3 to about 4, about 3 to about 5, about 3 to about 6, about 3 to about 7, about 3 to about 8, about 3 to about 9, about 3 to about 10, about 3 to about 11, about 3 to about 12, about 4 to about 5, about 4 to about 6, about 4 to about 7, about 4 to about 8, about 4 to about 9, about 4 to about 10, about 4 to about 11, about 4 to about 12, about 5 to about 6, about 5 to about 7, about 5 to about 8, about 5 to about 9, about 5 to about 10, about 5 to about 11, about 5 to about 12, about 6 to about 7, about 6 to about 8, about 6 to about 9, about 6 to about 10, about 6 to about 11, about 6 to about 12, about 7 to about 8, about 7 to about 9, about 7 to about 10, about 7 to about 11, about 7 to about 12, about 8 to about 9, about 8 to about 10, about 8 to about 11, about 8 to about 12, about 9 to about 10, about 9 to about 11, about 9 to about 12, about 10 to about 11, about 10 to about 12, or about 11 to about 12. In some embodiments, the pH is about 1, about 2, about 3, about 4, about 5, about 6, about 7, about 8, about 9, about 10, about 11, or about 12. In some embodiments, the pH is at least about 1, about 2, about 3, about 4, about 5, about 6, about 7, about 8, about 9, about 10, or about 11. In some embodiments, the pH is at most about 2, about 3, about 4, about 5, about 6, about 7, about 8, about 9, about 10, about 11, or about 12.

In some embodiments, the one or more environmental triggers comprises temperature. In some embodiments, the temperature is about −10 degrees Celsius to about 100 degrees Celsius. In some embodiments, the temperature is about −10 degrees Celsius to about 0 degrees Celsius, about −10 degrees Celsius to about 10 degrees Celsius, about −10 degrees Celsius to about 20 degrees Celsius, about −10 degrees Celsius to about 30 degrees Celsius, about −10 degrees Celsius to about 40 degrees Celsius, about −10 degrees Celsius to about 50 degrees Celsius, about −10 degrees Celsius to about 60 degrees Celsius, about −10 degrees Celsius to about 70 degrees Celsius, about −10 degrees Celsius to about 80 degrees Celsius, about −10 degrees Celsius to about 90 degrees Celsius, about −10 degrees Celsius to about 100 degrees Celsius, about 0 degrees Celsius to about 10 degrees Celsius, about 0 degrees Celsius to about 20 degrees Celsius, about 0 degrees Celsius to about 30 degrees Celsius, about 0 degrees Celsius to about 40 degrees Celsius, about 0 degrees Celsius to about 50 degrees Celsius, about 0 degrees Celsius to about 60 degrees Celsius, about 0 degrees Celsius to about 70 degrees Celsius, about 0 degrees Celsius to about 80 degrees Celsius, about 0 degrees Celsius to about 90 degrees Celsius, about 0 degrees Celsius to about 100 degrees Celsius, about 10 degrees Celsius to about 20 degrees Celsius, about 10 degrees Celsius to about 30 degrees Celsius, about 10 degrees Celsius to about 40 degrees Celsius, about 10 degrees Celsius to about 50 degrees Celsius, about 10 degrees Celsius to about 60 degrees Celsius, about 10 degrees Celsius to about 70 degrees Celsius, about 10 degrees Celsius to about 80 degrees Celsius, about 10 degrees Celsius to about 90 degrees Celsius, about 10 degrees Celsius to about 100 degrees Celsius, about 20 degrees Celsius to about 30 degrees Celsius, about 20 degrees Celsius to about 40 degrees Celsius, about 20 degrees Celsius to about 50 degrees Celsius, about 20 degrees Celsius to about 60 degrees Celsius, about 20 degrees Celsius to about 70 degrees Celsius, about 20 degrees Celsius to about 80 degrees Celsius, about 20 degrees Celsius to about 90 degrees Celsius, about 20 degrees Celsius to about 100 degrees Celsius, about 30 degrees Celsius to about 40 degrees Celsius, about 30 degrees Celsius to about 50 degrees Celsius, about 30 degrees Celsius to about 60 degrees Celsius, about 30 degrees Celsius to about 70 degrees Celsius, about 30 degrees Celsius to about 80 degrees Celsius, about 30 degrees Celsius to about 90 degrees Celsius, about 30 degrees Celsius to about 100 degrees Celsius, about 40 degrees Celsius to about 50 degrees Celsius, about 40 degrees Celsius to about 60 degrees Celsius, about 40 degrees Celsius to about 70 degrees Celsius, about 40 degrees Celsius to about 80 degrees Celsius, about 40 degrees Celsius to about 90 degrees Celsius, about 40 degrees Celsius to about 100 degrees Celsius, about 50 degrees Celsius to about 60 degrees Celsius, about 50 degrees Celsius to about 70 degrees Celsius, about 50 degrees Celsius to about 80 degrees Celsius, about 50 degrees Celsius to about 90 degrees Celsius, about 50 degrees Celsius to about 100 degrees Celsius, about 60 degrees Celsius to about 70 degrees Celsius, about 60 degrees Celsius to about 80 degrees Celsius, about 60 degrees Celsius to about 90 degrees Celsius, about 60 degrees Celsius to about 100 degrees Celsius, about 70 degrees Celsius to about 80 degrees Celsius, about 70 degrees Celsius to about 90 degrees Celsius, about 70 degrees Celsius to about 100 degrees Celsius, about 80 degrees Celsius to about 90 degrees Celsius, about 80 degrees Celsius to about 100 degrees Celsius, or about 90 degrees Celsius to about 100 degrees Celsius. In some embodiments, the temperature is about −10 degrees Celsius, about 0 degrees Celsius, about 10 degrees Celsius, about 20 degrees Celsius, about 30 degrees Celsius, about 40 degrees Celsius, about 50 degrees Celsius, about 60 degrees Celsius, about 70 degrees Celsius, about 80 degrees Celsius, about 90 degrees Celsius, or about 100 degrees Celsius. In some embodiments, the temperature is at least about −10 degrees Celsius, about 0 degrees Celsius, about 10 degrees Celsius, about 20 degrees Celsius, about 30 degrees Celsius, about 40 degrees Celsius, about 50 degrees Celsius, about 60 degrees Celsius, about 70 degrees Celsius, about 80 degrees Celsius, or about 90 degrees Celsius. In some embodiments, the temperature is at most about 0 degrees Celsius, about 10 degrees Celsius, about 20 degrees Celsius, about 30 degrees Celsius, about 40 degrees Celsius, about 50 degrees Celsius, about 60 degrees Celsius, about 70 degrees Celsius, about 80 degrees Celsius, about 90 degrees Celsius, or about 100 degrees Celsius.

In some embodiments, the one or more environmental triggers comprises salt concentration. The salt concentration may comprise the concentration of one salt. The salt concentration may comprise the cumulative concentration of multiple salts. A salt may comprise a food ingredient as defined herein, wherein the food ingredient dissociates into positive and negative ionic components such that the electrical conductivity of an aqueous solution is increased upon dissolution of said salt therein.

In some embodiments, the salt concentration is equal to or greater than about 10 mM to about 600 mM. In some embodiments, the salt concentration is equal to or greater than about 10 mM to about 25 mM, about 10 mM to about 50 mM, about 10 mM to about 100 mM, about 10 mM to about 150 mM, about 10 mM to about 200 mM, about 10 mM to about 250 mM, about 10 mM to about 300 mM, about 10 mM to about 350 mM, about 10 mM to about 400 mM, about 10 mM to about 500 mM, about 10 mM to about 600 mM, about 25 mM to about 50 mM, about 25 mM to about 100 mM, about 25 mM to about 150 mM, about 25 mM to about 200 mM, about 25 mM to about 250 mM, about 25 mM to about 300 mM, about 25 mM to about 350 mM, about 25 mM to about 400 mM, about 25 mM to about 500 mM, about 25 mM to about 600 mM, about 50 mM to about 100 mM, about 50 mM to about 150 mM, about 50 mM to about 200 mM, about 50 mM to about 250 mM, about 50 mM to about 300 mM, about 50 mM to about 350 mM, about 50 mM to about 400 mM, about 50 mM to about 500 mM, about 50 mM to about 600 mM, about 100 mM to about 150 mM, about 100 mM to about 200 mM, about 100 mM to about 250 mM, about 100 mM to about 300 mM, about 100 mM to about 350 mM, about 100 mM to about 400 mM, about 100 mM to about 500 mM, about 100 mM to about 600 mM, about 150 mM to about 200 mM, about 150 mM to about 250 mM, about 150 mM to about 300 mM, about 150 mM to about 350 mM, about 150 mM to about 400 mM, about 150 mM to about 500 mM, about 150 mM to about 600 mM, about 200 mM to about 250 mM, about 200 mM to about 300 mM, about 200 mM to about 350 mM, about 200 mM to about 400 mM, about 200 mM to about 500 mM, about 200 mM to about 600 mM, about 250 mM to about 300 mM, about 250 mM to about 350 mM, about 250 mM to about 400 mM, about 250 mM to about 500 mM, about 250 mM to about 600 mM, about 300 mM to about 350 mM, about 300 mM to about 400 mM, about 300 mM to about 500 mM, about 300 mM to about 600 mM, about 350 mM to about 400 mM, about 350 mM to about 500 mM, about 350 mM to about 600 mM, about 400 mM to about 500 mM, about 400 mM to about 600 mM, or about 500 mM to about 600 mM. In some embodiments, the salt concentration is equal to or greater than about 10 mM, about 25 mM, about 50 mM, about 100 mM, about 150 mM, about 200 mM, about 250 mM, about 300 mM, about 350 mM, about 400 mM, about 500 mM, or about 600 mM. In some embodiments, the salt concentration is equal to or greater than at least about 10 mM, about 25 mM, about 50 mM, about 100 mM, about 150 mM, about 200 mM, about 250 mM, about 300 mM, about 350 mM, about 400 mM, or about 500 mM. In some embodiments, the salt concentration is equal to or greater than at most about 25 mM, about 50 mM, about 100 mM, about 150 mM, about 200 mM, about 250 mM, about 300 mM, about 350 mM, about 400 mM, about 500 mM, or about 600 mM.

In some embodiments, the one or more environmental triggers comprise food additive concentration. The salt concentration may comprise the concentration of one food additive. The salt concentration may comprise the cumulative concentration of multiple food additives.

In some embodiments, the food additive concentration is equal to or greater than about 10 mM to about 600 mM. In some embodiments, the food additive concentration is equal to or greater than about 10 mM to about 25 mM, about 10 mM to about 50 mM, about 10 mM to about 100 mM, about 10 mM to about 150 mM, about 10 mM to about 200 mM, about 10 mM to about 250 mM, about 10 mM to about 300 mM, about 10 mM to about 350 mM, about 10 mM to about 400 mM, about 10 mM to about 500 mM, about 10 mM to about 600 mM, about 25 mM to about 50 mM, about 25 mM to about 100 mM, about 25 mM to about 150 mM, about 25 mM to about 200 mM, about 25 mM to about 250 mM, about 25 mM to about 300 mM, about 25 mM to about 350 mM, about 25 mM to about 400 mM, about 25 mM to about 500 mM, about 25 mM to about 600 mM, about 50 mM to about 100 mM, about 50 mM to about 150 mM, about 50 mM to about 200 mM, about 50 mM to about 250 mM, about 50 mM to about 300 mM, about 50 mM to about 350 mM, about 50 mM to about 400 mM, about 50 mM to about 500 mM, about 50 mM to about 600 mM, about 100 mM to about 150 mM, about 100 mM to about 200 mM, about 100 mM to about 250 mM, about 100 mM to about 300 mM, about 100 mM to about 350 mM, about 100 mM to about 400 mM, about 100 mM to about 500 mM, about 100 mM to about 600 mM, about 150 mM to about 200 mM, about 150 mM to about 250 mM, about 150 mM to about 300 mM, about 150 mM to about 350 mM, about 150 mM to about 400 mM, about 150 mM to about 500 mM, about 150 mM to about 600 mM, about 200 mM to about 250 mM, about 200 mM to about 300 mM, about 200 mM to about 350 mM, about 200 mM to about 400 mM, about 200 mM to about 500 mM, about 200 mM to about 600 mM, about 250 mM to about 300 mM, about 250 mM to about 350 mM, about 250 mM to about 400 mM, about 250 mM to about 500 mM, about 250 mM to about 600 mM, about 300 mM to about 350 mM, about 300 mM to about 400 mM, about 300 mM to about 500 mM, about 300 mM to about 600 mM, about 350 mM to about 400 mM, about 350 mM to about 500 mM, about 350 mM to about 600 mM, about 400 mM to about 500 mM, about 400 mM to about 600 mM, or about 500 mM to about 600 mM. In some embodiments, the food additive concentration is equal to or greater than about 10 mM, about 25 mM, about 50 mM, about 100 mM, about 150 mM, about 200 mM, about 250 mM, about 300 mM, about 350 mM, about 400 mM, about 500 mM, or about 600 mM. In some embodiments, the food additive concentration is equal to or greater than at least about 10 mM, about 25 mM, about 50 mM, about 100 mM, about 150 mM, about 200 mM, about 250 mM, about 300 mM, about 350 mM, about 400 mM, or about 500 mM. In some embodiments, the food additive concentration is equal to or greater than at most about 25 mM, about 50 mM, about 100 mM, about 150 mM, about 200 mM, about 250 mM, about 300 mM, about 350 mM, about 400 mM, about 500 mM, or about 600 mM.

### Physicochemical Properties

In some embodiments, the method comprises computer processing the primary amino acid sequence of the candidate protein to determine a set of physicochemical properties of the candidate protein. The set of physicochemical properties of the candidate protein may correlate to the presence of a target food function in the candidate protein. The set of physicochemical properties of the candidate protein may correlate to the degree of a target food function in the candidate protein. The set of physicochemical properties of the candidate protein may correlate to the absence of a target food function in the candidate protein. In some embodiments, the method comprises computer processing the set of physicochemical properties of the candidate protein to provide an output that is indicative of whether the candidate protein has the target food function.

In some embodiments, the set of physicochemical properties of the candidate protein comprises sequence length, molecular weight, isoelectric point, pH-adjusted net charge per residue, a presence or number of hydrophobic amino acids within the sequence, a presence or number of aliphatic amino acids within the sequence, a presence or number of aromatic amino acids within the sequence, a presence or number of positively charged amino acids within the sequence, a presence or number of negatively charged amino acids within the sequence, a presence or number of polar amino acids within the sequence, a presence or number of glycine amino acids within the sequence, a presence or number of alanine amino acids within the sequence, a presence or number of cysteine amino acids within the sequence, a presence or number of proline amino acids within the sequence, a presence or number of histidine amino acids within the sequence, a distribution of hydrophobic amino acids within the sequence, a distribution of aliphatic amino acids within the sequence, a distribution of aromatic amino acids within the sequence, a distribution of positively charged amino acids within the sequence, a distribution of negatively charged amino acids within the sequence, a distribution of polar amino acids within the sequence, a distribution of glycine amino acids within the sequence, a distribution of alanine amino acids within the sequence, a distribution of cysteine amino acids within the sequence, a distribution of proline amino acids within the sequence, and/or a distribution of histidine amino acids within the sequence. In some embodiments, the sequence is the primary amino acid sequence of the candidate protein.

In some embodiments, the set of physicochemical properties of the candidate protein comprises a presence or number of clustering motifs within the sequence and/or a distribution of clustering motifs within the sequence. A clustering motif includes any peptide sequence of two or more amino acids that is present twice or more within the primary amino acid sequence. In some embodiments, the amino acid comprising the two or more clustering motifs are identical in sequence in all instances. In some embodiments, the amino acid comprising the two or more clustering motifs have similar physicochemical properties. In some embodiments, the clustering motif is a dipeptide motif. In some embodiments, the clustering motif comprises two or more amino acids within a primary amino acid sequence that are adjacent or non-adjacent to one another. In some embodiments, the presence or number is determined based upon a single distinct clustering motif. In some embodiments, the distribution is determined based upon a single distinct clustering motif. In some embodiments, the presence or number is determined based upon multiple distinct clustering motifs. In some embodiments, the distribution is determined based upon multiple distinct clustering motifs.

In some embodiments, the distribution of a specified feature is expressed in terms of the mean inverse distance weight parameter of amino acids or another specified feature (e.g., a clustering motif, a dipeptide motif) within the sequence. The mean inverse distance weight parameter may be expressed as a quantitative value, wherein the quantitative value is a number equal to or greater than zero. In some embodiments, the distribution is determined based upon the complete primary amino acid sequence of the candidate protein. In some embodiments, the distribution is determined based upon a fragment of the primary amino acid sequence of the candidate protein.

In some embodiments, the set of physicochemical properties comprises one or more normalized sequence asymmetry parameters. A normalized sequence asymmetry parameter may be expressed as a numerical value in the inclusive range of −1 to 1. In some embodiments, a normalized sequence asymmetry parameter is utilized to determine the relative prevalence of a first feature in a candidate protein (e.g., the primary amino acid sequence of the candidate protein) versus the prevalence of a second feature in the candidate protein. A normalized sequence asymmetry parameter may provide a means of referencing the presence or number of one feature (e.g., an amino acid, a clustering motif) against the presence or number of another feature. In some embodiments, a normalized sequence asymmetry parameter is determined based upon the complete primary amino acid sequence of the candidate protein. In some embodiments, a normalized sequence asymmetry parameter is determined based upon a fragment of the primary amino acid sequence of the candidate protein.

In some embodiments, the set of physicochemical properties comprises a normalized sequence asymmetry parameter between aromatic amino acids and aliphatic amino acids. In some embodiments, the set of physicochemical properties comprises a normalized sequence asymmetry parameter between lysine amino acids and arginine amino acids. In some embodiments, the set of physicochemical properties comprises one or more normalized sequence asymmetry parameters between a first feature and a second feature, wherein: the first feature is an amino acid or clustering motif as described herein; and the second feature an amino acid or clustering motif as described herein that is not the first feature.

In some embodiments, the candidate protein or individual protein comprises a normalized sequence asymmetry parameter between lysine amino acids and arginine amino acids that is biased towards lysine amino acids. In some embodiments, the candidate protein or individual protein comprises a normalized sequence asymmetry parameter between lysine amino acids and arginine amino acids that is biased towards arginine amino acids. In some embodiments, the candidate protein or individual protein comprises a normalized sequence asymmetry parameter between aromatic amino acids and aliphatic amino acids that is biased towards aromatic amino acids. In some embodiments, the candidate protein or individual protein comprises a normalized sequence asymmetry parameter between aromatic amino acids and aliphatic amino acids that is biased towards aliphatic amino acids.

In some embodiments, the candidate protein or individual protein comprises a pH-adjusted net charge per residue in the inclusive range of +/−0.01 to 0.20. In some embodiments, the candidate protein or individual protein comprises a pH-adjusted net charge per residue in the inclusive range of +/−0.01 to 0.15. In some embodiments, the candidate protein or individual protein comprises a pH-adjusted net charge per residue in the inclusive range of +/−0.01 to 0.10. In some embodiments, the candidate protein or individual protein comprises a pH-adjusted net charge per residue in the inclusive range of +/−0.05 to 0.20. In some embodiments, the candidate protein or individual protein comprises a pH-adjusted net charge per residue in the inclusive range of +/−0.02 to 0.08. In some embodiments, the candidate protein or individual protein comprises a pH-adjusted net charge per residue in the inclusive range of +/−0.02 to 0.07. In some embodiments, the candidate protein or individual protein comprises a pH-adjusted net charge per residue in the inclusive range of +/−0.03 to 0.08. In some embodiments, the candidate protein or individual protein comprises a pH-adjusted net charge per residue in the inclusive range of +/−0.03 to 0.07. In some embodiments, the candidate protein or individual protein comprises a pH-adjusted net charge per residue in the inclusive range of +/−0.03 to 0.06. In some embodiments, the candidate protein or individual protein comprises a pH-adjusted net charge per residue in the inclusive range of +/−0.04 to 0.07.

In some embodiments, the candidate protein or individual protein comprises a distribution of hydrophobic amino acids that is below 3.0. In some embodiments, the candidate protein or individual protein comprises a distribution of aromatic amino acids that is below 1.5. In some embodiments, the candidate protein or individual protein comprises a distribution of aliphatic amino acids that is below 1.5. In some embodiments, the candidate protein or individual protein comprises a distribution of positively charged amino acids that is below 1.25. In some embodiments, the candidate protein or individual protein comprises a distribution of negatively charged amino acids that is below 1.5.

In some embodiments of the method of predicting a target food function of a candidate protein, the method comprises computer processing the set of candidate amino acid sequences such that the set of candidate amino acid sequences comprises only candidate amino acid sequences with one or more physicochemical properties of about a certain predetermined threshold value or within a certain range of predetermined threshold values.

Candidate proteins predicted and evaluated to have a target food function by the methods described herein can be incorporated into a composition comprising an individual protein as described herein. Candidate proteins predicted and evaluated to have a target food function by the methods described herein can be incorporated into a food product comprising the candidate protein and one or more food ingredients. The food product may be produced and sold commercially.

### Methods

In an aspect, disclosed here is a method of predicting a target food function of a candidate protein, the method comprising: (a) providing an amino acid sequence for the candidate protein; (b) computer processing the amino acid sequence of the candidate protein to predict a set of candidate amino acid sequences having intrinsic disorder; and (c) computer processing the set of candidate amino acid sequences to generate an output that is indicative of whether the candidate protein has the target food function. In some embodiments, the method further comprises computer processing the set of candidate amino acid sequences to determine a set of physicochemical properties of individual candidate amino acid sequences. In some embodiments, the method further comprises computer processing of the set of physicochemical properties to generate the output.

In an aspect, disclosed herein is a method of predicting a target food function of a candidate protein, the method comprising: (a) providing an amino acid sequence for the candidate protein; (b) computer processing the amino acid sequence of the candidate protein to generate a set of candidate amino acid sequences and a set of physicochemical properties of individual candidate amino acid sequences; and (c) computer processing the set of candidate amino acid sequences and the set of physicochemical properties to generate an output that is indicative of whether the candidate protein has the target food function. In some embodiments, the method further comprises computer processing the set of candidate amino acid sequences to predict a set of candidate amino acid sequences having intrinsic disorder. In some embodiments, the method comprises computer processing of the set of candidate amino acid sequences having intrinsic disorder to generate the output.

In some embodiments, the computer processing comprises using a trained computer model. In some embodiments, the trained computer model comprises a trained machine learning model. In some embodiments, the machine learning computer model comprises a recurrent neural network. In some embodiments, the machine learning computer model comprises a transformer protein language model. In some embodiments, the trained computer model is trained using a training data set comprising: a set of amino acid sequences of individual proteins with the target food function; and/or a set of amino acid sequences of individual proteins without the target food function. In some embodiments, the trained computer model is trained using a training data set comprising: a set of physicochemical properties of a set of amino acid sequences of individual proteins with the target food function; and/or a set of physicochemical properties of a set of amino acid sequences of individual proteins without the target food function. In some embodiments, the trained computer model is trained using a training data set comprising: a set of amino acid sequences of traditional food proteins, and/or a set of physicochemical properties a set of amino acid sequences of traditional food proteins. In some embodiments, the trained computer model is trained using a training data set comprising: a set of amino acid sequences of naturally occurring and/or synthetic variants of traditional food proteins, and/or a set of physicochemical properties a set of amino acid sequences of naturally occurring and/or synthetic variants of traditional food proteins. In some embodiments, the set of physicochemical properties is determined by analytical assays. In some embodiments, the set of physicochemical properties is determined by computer processing the amino acid sequences. A synthetic protein (e.g., a candidate protein that is synthetic) and/or a synthetic variant (e.g., the primary amino acid sequence of a synthetic protein and/or a synthetic variant) may be provided by computer processing (e.g., optimization of a primary amino acid sequence).

In some embodiments, the method comprises recombinantly expressing one or more candidate amino acid sequences within the set of candidate amino acid sequences to provide quantities of candidate proteins. In some embodiments, the method comprises extracting and/or purifying one or more candidate amino acid sequences within the set of candidate amino acid sequences from their natural source (e.g., plants, fungi, protists) to provide quantities of candidate proteins. In some embodiments, the quantity of the candidate protein provided by said recombinant expression can be in the inclusive range of 0.01 mg to 100 kg. In some embodiments, the quantity of the candidate protein provided by said recombinant expression can be in the inclusive range of 1 mg to 10 kg. In some embodiments, the quantity of the candidate protein provided by said recombinant expression can be in the inclusive range of 10 mg to 1 kg. In some embodiments, the quantity of the candidate protein provided by said recombinant expression can be in the inclusive range of 10 mg to 100 g. In some embodiments, the quantity of the candidate protein provided by said recombinant expression can be in the inclusive range of 10 mg to 1 g. In some embodiments, the quantity of the candidate protein provided by said recombinant expression can be in the inclusive range of 1 g to 100 g. In some embodiments, the quantity of the candidate protein provided by said recombinant expression can be in the inclusive range of 1 g to 10 g. In some embodiments, the quantity of the candidate protein provided by said recombinant expression can be in the inclusive range of 10 g to 100 g.

In some embodiments, recombinant expression of the candidate protein may comprise recombinant expression of the candidate protein in addition to a tag sequence or reporter sequence incorporated into the primary amino acid sequence of the candidate protein. The tag sequence or the reporter sequence may be utilized in the purification or isolation of the candidate protein. The tag sequence or the reporter sequence may be utilized in the analytical assays. The tag sequence or the reporter sequence may be selected to enhance the ability to purify or isolate the candidate protein by chromatography (e.g., immobilized metal affinity chromatography, normal phase chromatography, reverse phase chromatography, size exclusion chromatography). The tag sequence or the reporter sequence may be selected for a spectroscopic property (e.g., absorption) that provides the basis for measurement of one or more analytical assays. The tag sequence or the reporter sequence may be selected for a photophysical property (e.g., fluorescence, luminescence) that provides the basis for measurement of one or more analytical assays. In some embodiments, the tag sequence or the reporter sequence comprises amino acids. In some embodiments, the tag sequence or the reporter sequence comprises amino acids wherein one or more amino acids are functionalized or conjugated with one or more chemical functionalities that enable the intended function of the tag sequence or the reporter sequence. In some embodiments, the tag sequence or reporter sequence comprises a hemagglutinin (HA) tag. In some embodiments, the tag sequence or reporter sequence comprises a TEV cleavage site (e.g., a cleavage site that may be cleaved by tobacco etch virus protease). In some embodiments, the tag sequence or reporter sequence comprises a His18 tag. In some embodiments, the tag sequence or reporter sequence comprises an amino acid sequence of SEQ ID NO.: 44 In some embodiments, the tag sequence or reporter sequence comprises an amino acid sequence of SEQ ID NO.: 45. In some embodiments, the tag sequence or reporter sequence comprises an amino acid sequence of SEQ ID NO.: 46.

According to some embodiments, SEQ ID NO: 44 consists of the primary amino acid sequence: MKHHSSHHGHHGHHGHHGHHGHHGHHGHH.

According to some embodiments, SEQ ID NO: 45 consists of the primary amino acid sequence:

MKHHSSHHGHHGHHGHHGHHGHHGHHGHHGTYPYDVPDYAGTSENLYFQG.

According to some embodiments, SEQ ID NO: 46 consists of the primary amino acid sequence:

MKHHSSHHGHHGHHGHHGHHGHHGHHGHHGTSAAGGEEDKKPAGGEGGG
AHINLKVKGQDGNEVFFRIKRSTQLKKLMNAYCDRQSVDMTAIAFLFDG
RRLRAEQTPDELEMEDGDEIDAMLHQTGGGS.

In some embodiments, a protein comprising the amino acid sequence SEQ ID NO: 44 can be expressed using a polynucleotide sequence that comprises SEQ ID NO: 47.

According to some embodiments, SEQ ID NO: 47 consists of the polynucleotide sequence:

ATGAAACATCATAGTAGTCACCACGGTCATCACGGTCATCACGGTCATC
ACGGTCATCACGGGCATCACGGTCATCACGGTCATCAC.

In some embodiments, a protein comprising the amino acid sequence SEQ ID NO: 45 can be expressed using a polynucleotide sequence that comprises SEQ ID NO: 48.

According to some embodiments, SEQ ID NO: 48 consists of the polynucleotide sequence:

ATGAAACATCATAGTAGTCACCACGGTCATCACGGTCATCACGGTCATC
ACGGTCATCACGGGCATCACGGTCATCACGGTCATCACGGTACCTACCC
TTACGACGTGCCAGACTACGCCGGAACTAGTGAGAACCTGTATTTTCAA
GGA.

In some embodiments, a protein comprising the amino acid sequence SEQ ID NO: 46 can be expressed using a polynucleotide sequence that comprises SEQ ID NO: 49.

According to some embodiments, SEQ ID NO: 49 consists of the polynucleotide sequence:

ATGAAACATCATAGTAGTCACCACGGTCATCACGGTCATCACGGTCATC
ACGGTCATCACGGGCATCACGGTCATCACGGTCATCACGGTACCTCTGC
TGCGGGTGGCGAAGAAGATAAGAAACCGGCAGGTGGCGAAGGTGGCGGT
GCCCATATCAACCTGAAAGTGAAAGGTCAAGACGGCAACGAAGTCTTTT
TCCGCATCAAACGTTCTACCCAGCTGAAAAAGCTGATGAACGCATACTG
TGACCGTCAGTCTGTAGACATGACCGCAATTGCTTTCCTGTTTGATGGT
CGTCGCCTGCGTGCGGAACAGACCCCGGATGAACTGGAGATGGAAGATG
GCGACGAAATCGACGCAATGCTGCACCAGACTGGTGGCGGATCC.

In some embodiments, the method comprises conducting analytical assays to determine whether the candidate proteins have the target food function or do not have the target food function. The analytical assays can provide an experimental validation of the target food function or one or more physicochemical properties of the candidate protein. In some embodiments, the analytical assays measure the concentration of the candidate protein in solution. In some embodiments, the analytical assays measure the concentration of the candidate protein in solution as a variable function of an environmental condition. In some embodiments, the environmental condition is temperature, pH, salt concentration, food additive concentration, the presence of one or more enzymes, or combinations thereof. In some embodiments, a set of environmental conditions comprises multiple environmental conditions wherein the environmental conditions are at independent and discrete values.

In some embodiments, the analytical assays provide an experimental measurement of the target food function in the candidate protein. In some embodiments, the analytical assays provide a determination that the target food function in the candidate protein depends on a single environmental condition. In some embodiments, the analytical assays provide a determination that the target food function in the candidate protein depends on multiple environmental conditions (e.g., a set of environmental conditions. In some embodiments, the analytical assays provide an experimental determination of the environmental trigger associated with the target food function in the candidate protein.

In some embodiments, the analytical assays comprise density measurements. In some embodiments, the analytical assays comprise concentration measurements. In some embodiments, the analytical assays comprise rheology measurements. In some embodiments, the analytical assays comprise dynamic light scattering measurements. In some embodiments, the analytical assays comprise viscometry measurements. In some embodiments, the analytical assays comprise differential scanning calorimetry measurements. In some embodiments, the analytical assays comprise colorimetric measurements. In some embodiments, the analytical assays comprise nuclear magnetic resonance spectroscopy. In some embodiments, the analytical assays comprise photospectrometry. In some embodiments, the analytical assays comprise fluorescence measurements. In some embodiments, the analytical assays comprise luminescence measurements. In some embodiments, the analytical assays comprise mass spectrometry. In some embodiments, the analytical assays comprise pH measurements. In some embodiments, the analytical assays comprise chromatography. In some embodiments, the analytical assays comprise gel electrophoresis. In some embodiments, the analytical assays comprise Western blots. In some embodiments, the analytical assays comprise conductivity measurements. In some embodiments, the analytical assays comprise circular dichroism measurements. In some embodiments, the analytical assays comprise melting temperature measurements. In some embodiments, the analytical assays comprise denaturing temperature measurements. In some embodiments, the analytical assays comprise texture profile analysis. In some embodiments, a texture profile obtained from texture profile analysis may comprise one or more mechanical properties (e.g., density, elasticity, plasticity, response to compressive force, response to shear force, etc.).

In some embodiments, analytical assays are conducted on the light phase. In some embodiments, analytical assays are conducted on the dense phase. In some embodiments, the analytical assays comprise standard laboratory methods. In some embodiments, the analytical assays comprise methods developed in-house. In some embodiments, the target food function comprises a texture profile or a property associated with the texture profile.

In some embodiments, the target food function is quantitatively associated with the measurements conducted in the analytic assays. In some embodiments, the degree of the target food function is proportional to a measurement conducted in the analytic assays. In some embodiments, the degree of the target food function is inversely proportional to a measurement conducted in the analytic assays.

In some embodiments, the analytical assays comprise measuring the concentration in the light phase. In some embodiments, the light phase is an aqueous solution. In some embodiments, the degree of the target food function is proportional or inversely proportional to the concentration of the candidate protein aqueous solution under a predetermined set of environmental conditions.

In some embodiments, the target food function is a concentration of the candidate protein in aqueous solution above or below a predetermined threshold. In some embodiments, the predetermined threshold is in the inclusive range of 1 micromolar to 1 molar. In some embodiments, the predetermined threshold is in the inclusive range of 10 micromolar (μM) to 0.1 molar (M). In some embodiments, the predetermined threshold is in the inclusive range of 100 micromolar to 0.1 molar. In some embodiments, the predetermined threshold is in the inclusive range of 1 millimolar (mM) to 1 molar. In some embodiments, the predetermined threshold is in the inclusive range of 10 micromolar (μM) to 100 millimolar (mM). In some embodiments, the predetermined threshold is in the inclusive range of 100 millimolar to 1 molar. In some embodiments, the predetermined threshold is 10 mM. In some embodiments, the predetermined threshold is 5 mM. In some embodiments, the predetermined threshold is 1 mM. In some embodiments, the predetermined threshold is 500 μM. In some embodiments, the predetermined threshold is 250 μM. In some embodiments, the predetermined threshold is 100 μM. In some embodiments, the predetermined threshold is 50 μM. In some embodiments, the predetermined threshold is 25 μM. In some embodiments, the predetermined threshold is 10 μM. In some embodiments, the predetermined threshold is 5 μM. In some embodiments, the predetermined threshold is 1 μM.

### Compositions

In an aspect, disclosed herein are compositions comprising: a candidate protein predicted and/or evaluated to have a target food function by the methods disclosed herein; and one or more food ingredients. In a further aspect, disclosed herein are food products comprising a candidate protein predicted and/or evaluated to have a target food function by the methods disclosed herein.

In an aspect, disclosed herein are compositions comprising: an individual protein, wherein the individual protein comprises a primary amino acid sequence that is conserved by 50% or more relative to any one of SEQ ID NOs: 1 to 43, and one or more food ingredients. In some embodiments, the individual protein comprises a primary amino acid sequence that is conserved by 60% or more relative to any one of SEQ ID NOs: 1 to 43. In some embodiments, the individual protein comprises a primary amino acid sequence that is conserved by 70% or more relative to any one of SEQ ID NOs: 1 to 43. In some embodiments, the individual protein comprises a primary amino acid sequence that is conserved by 80% or more relative to any one of SEQ ID NOs: 1 to 43. In some embodiments, the individual protein comprises a primary amino acid sequence that is conserved by 90% or more relative to any one of SEQ ID NOs: 1 to 43. In some embodiments, the individual protein comprises a primary amino acid sequence that is any one of SEQ ID NOs: 1 to 43.

In some embodiments, the individual protein has intrinsic disorder. In some embodiments, intrinsic disorder comprises a lack of stable secondary structure along at least about 10% of the length of the primary amino acid sequence of the individual protein. In some embodiments, intrinsic disorder comprises a lack of stable secondary structure along at least about 30% of the length of the primary amino acid sequence of the individual protein. In some embodiments, intrinsic disorder comprises a lack of stable secondary structure along at least about 50% of the length of the primary amino acid sequence of the individual protein. In some embodiments, intrinsic disorder comprises a lack of stable secondary structure along at least about 75% of the length of the primary amino acid sequence of the individual protein. In some embodiments, intrinsic disorder comprises a lack of stable secondary structure along at least about 90% of the length of the primary amino acid sequence of the individual protein.

In some embodiments, the individual protein has a target food function. In some embodiments, the target food function satisfies a predetermined criterion. In some embodiments, the predetermined criterion comprises the target food function being above a predetermined threshold. In some embodiments, the predetermined criterion comprises the target food function being below a predetermined threshold.

In some embodiments, the individual protein has physicochemical properties to an identical or improved degree as compared to the degree to which a protein with a primary amino acid sequence consisting of any one of SEQ ID NOs: 1 to 43 has physicochemical properties; wherein the protein with a primary amino acid sequence consisting of any one of SEQ ID NOs: 1 to 43 is that with the greatest conservation relative to the individual protein. In some embodiments, the physicochemical properties are correlated to the target food function such that the physicochemical properties affect the degree to which the individual protein has the target food function.

In some embodiments, the individual protein was predicted to have a target food function according to the methods disclosed herein.

In an aspect, disclosed herein are food products comprising a composition as disclosed herein.

In some embodiments, the individual protein comprises a primary amino acid sequence that is conserved by 50% or more relative to SEQ ID NO: 1.

According to some embodiments, SEQ ID NO: 1 consists of the primary amino acid sequence:

MEFQQAAVEANKPVQHQQQQIMDYFSFFEEDQPASTLLQLAAPVLPHLQ
LAQPNIQDSP.

In some embodiments, the individual protein comprises a primary amino acid sequence that is conserved by 50% or more relative to SEQ ID NO: 2.

According to some embodiments, SEQ ID NO: 2 consists of the primary amino acid sequence:

MPLNNNTPSYSHPRNSNIIFNFILHLLTTTSSSILSKSQNLSQLVDQEN
KDVDLVVSSSFVNVEELSLIQSDSKPK.

In some embodiments, the individual protein comprises a primary amino acid sequence that is conserved by 50% or more relative to SEQ ID NO: 3.

According to some embodiments, SEQ ID NO: 3 consists of the primary amino acid sequence:

MSTELVSIGQNQVPNISNIGESNSVLTNVDAPPIQDETHFSSPTDVEEA
RPKQISSIWNHFE.

In some embodiments, the individual protein comprises a primary amino acid sequence that is conserved by 50% or more relative to SEQ ID NO: 4.

According to some embodiments, SEQ ID NO: 4 consists of the primary amino acid sequence:

MSNENEYVDEKKVKPLACFSSPIVVSSSYPQHSLFEAPSFSLGPEFEDD
PETEDEKPVIKEICIRQNPRRDVGVAAHSRSPFKFRQILPNVPIKPSEE
RVSDILFMIPEETNQV.

In some embodiments, the individual protein comprises a primary amino acid sequence that is conserved by 50% or more relative to SEQ ID NO: 5.

According to some embodiments, SEQ ID NO: 5 consists of the primary amino acid sequence:

MNNQPPSPNNVLDNFILPSSDTNSLIANYGSEEIVNNNVVPQAPTKSVG
MGNGWFMQNNQQQYPMINLEELMQDDVWSNPQNDQVTTSPFRGIGDEEV
WE.

In some embodiments, the individual protein comprises a primary amino acid sequence that is conserved by 50% or more relative to SEQ ID NO: 6.

According to some embodiments, SEQ ID NO: 6 consists of the primary amino acid sequence:

MVFPLPDERNWHNPGNLPKLKPPAMVKQQSGRPKNKDRFPSKYEEPISK
QCGRCGCKSHTREACMQPLPKSGNGKQPFDCSGVRKRKSDIEREVIIES
NPGNETEVINNPNMEVDEEYFENGRIYADWEDLD.

In some embodiments, the individual protein comprises a primary amino acid sequence that is conserved by 50% or more relative to SEQ ID NO: 7.

According to some embodiments, SEQ ID NO: 7 consists of the primary amino acid sequence:

MYWILNNLVWNRVDPIVEMGLGQPDSFVEEGEPQSYTIPLRFHSEKSSF
RKNQIQELSPEIRRN.

In some embodiments, the individual protein comprises a primary amino acid sequence that is conserved by 50% or more relative to SEQ ID NO: 8.

According to some embodiments, SEQ ID NO: 8 consists of the primary amino acid sequence:

MDQNNNQEMYSHSIQPTQITQQIILPPLTTTIIQQIFHPPFPTTITCMP
TTTAIPQEVIDLQQQPKKLDD.

In some embodiments, the individual protein comprises a primary amino acid sequence that is conserved by 50% or more relative to SEQ ID NO: 9.

According to some embodiments, SEQ ID NO: 9 consists of the primary amino acid sequence:

MHICKTSQSSIFISYENQKPNQCQIKKQKVGKLNTIKEETVPFTPLKCE
SQNAQFFVNSIEPFTSEYMYLPLLPNQEEEFEMDKIFQSSELDRIEFSS
MPTSIIESPFCREWDLSVYDQLDVHDPNSMINISEYFT.

In some embodiments, the individual protein comprises a primary amino acid sequence that is conserved by 50% or more relative to SEQ ID NO: 10.

According to some embodiments, SEQ ID NO: 10 consists of the primary amino acid sequence:

MGQEQNQNHHRQQAEQPQQPPLSVGISPEQHPLHPDDGYPLESRDSSNH
KRPSNNTSYEEFVAAEEERQRLILSEDMDSNGFPNSWPKVSPPPFLPVT
VAPPTVEPPSPEKDFSPPTVTRNIESPPLRPPPPPSKCCIIL.

In some embodiments, the individual protein comprises a primary amino acid sequence that is conserved by 50% or more relative to SEQ ID NO: 11.

According to some embodiments, SEQ ID NO: 11 consists of the primary amino acid sequence:

MADANNKEAQSKSKNPLEPRNFFSMLPRVEFKFPSFDQQSEKPDASVEK
EEQIEIAKPPSVFMGNRRKNPPPLEFEAEECLGRTSNPIVLWQVSQPYC
HSAEPYMRLSFNSFEVIGFFPRSRIFSPLPLCPEE.

In some embodiments, the individual protein comprises a primary amino acid sequence that is conserved by 50% or more relative to SEQ ID NO: 12.

According to some embodiments, SEQ ID NO: 12 consists of the primary amino acid sequence:

MLEEENNNMYRWIQEHRAAIEYQQHGGLEAKPVEHHQQVLDEFPFYGEP
SSVLQLATIPQQFSYQLQLAQPNLQDSNV.

In some embodiments, the individual protein comprises a primary amino acid sequence that is conserved by 50% or more relative to SEQ ID NO: 13.

According to some embodiments, SEQ ID NO: 13 consists of the primary amino acid sequence:

MELQNDNMYLRNKVAENERVQQQMDMLPSTATTTEYEVMPSYDSRNFLQ
VSIMQPNQHYTHQQRTALQLGRV.

In some embodiments, the individual protein comprises a primary amino acid sequence that is conserved by 50% or more relative to SEQ ID NO: 14.

According to some embodiments, SEQ ID NO: 14 consists of the primary amino acid sequence:

MGGRKCHAASQEERECFEAHHLRRFNSSSMTQRDHNLKELMSIPFPFNL
PDEVKDLRLQLPCPPPFIPRHSPHDFFTSSSEGEWWNQVRCCTAFEIAS
QEGNGKQQAEHDR.

In some embodiments, the individual protein comprises a primary amino acid sequence that is conserved by 50% or more relative to SEQ ID NO: 15.

According to some embodiments, SEQ ID NO: 15 consists of the primary amino acid sequence:

MELQNDNVFLRNKITENERTQQQMNILPSTSEYDILTPFDSRNFLQVLQ
PAQHYSQQQQQAGLQLGYGQLVMNTN.

In some embodiments, the individual protein comprises a primary amino acid sequence that is conserved by 50% or more relative to SEQ ID NO: 16.

According to some embodiments, SEQ ID NO: 16 consists of the primary amino acid sequence:

MEQQHVEPQIPVDLDGKVEDGRQQLVENDQQPIVAQDEQHPKCAIRKSI
WMTNYERLSKSKMEEGNG.

In some embodiments, the individual protein comprises a primary amino acid sequence that is conserved by 50% or more relative to SEQ ID NO: 17.

According to some embodiments, SEQ ID NO: 17 consists of the primary amino acid sequence:

MPETEEVIEEGRDNTIEPSPPSKQFGFEDVFTEIPNQSLSAEGVLNLEP
DPFMKQLPHRHNRGIPKPTYEPELSTKVKYPMSNYVSNHRLSESNKSFV
NQLSIVAIPNSVQEALADPRWKTVMNEGMKSL.

In some embodiments, the individual protein comprises a primary amino acid sequence that is conserved by 50% or more relative to SEQ ID NO: 18.

According to some embodiments, SEQ ID NO: 18 consists of the primary amino acid sequence:

MDVMFHEDSIYFSSESELQGEYHNEIQTFDYDYHISKEDEFGQSELVNQ
EVGELDMSGQQFGSEDVFTEIPNQLSSVEGVLNLEPDPFMKRLPHCHNR
GIPKPTYEPDLSTKVKYPMSNYVFNHRLSESNKSFVNQLSTVIIPNSV.

In some embodiments, the individual protein comprises a primary amino acid sequence that is conserved by 50% or more relative to SEQ ID NO: 19.

According to some embodiments, SEQ ID NO: 19 consists of the primary amino acid sequence:

MGTKVRNLPEYCSIRDHNEESSSCGWPLFYGDKTVTNGQYCHNYLSSATADACSVYD
KDILKQKMLEHEVTFKNQVFELHRLYRIQRNLMGEVNMKELHRNKIPVETSFTTGPLAS
QITSEDGKKWYTHSFPIGSSTCARPSISGFEGSHSPLDTNKGISKQAGKFPSPKESSSK
DVEVLKCRPSKARRRIFDLHLPADDYIDTQESEKLGDERKSGATLFLPDRNCNHDVNV
FHSNGGKTGSQQDSSRPEKSLKSRNGLADLNEPIEIEEINDASYVPLLNHNLYLGKTEC
SDLCAKQNSRFFGLSTDDLINSHHGTDSWARNNGYMENDGSGKGFVLSGLETGLAKS
NLKPIPHVFKQEQSLLSPQNMKDTLSMAHKPTSDCRTNQSKADLWWEKAVSSLDVSE
GNHEYSTNKYAESVVSSHRSSLFAIAPSPDLVKSWSHSPSSLEMPTSSLNQKTASAQT
PPWLNASGVLSRSSQSHQSNAILGSTWPQHINSKANPGFQCEVPLQNGFYPGSSSGC
KELSANISSISYDYLNHNNNDRKRIPERCSNDSAKYYESSNSNCNNKQSGKDINLNVLL
SNGSSNMLVTQSGPGIMEGEQKHEEQIAVLPWLRAKTACKNEMQNVARGLTTRELGF
SHVTSLSDKDEIGQGSSEKVMHNVTSGLCSNDNEPRRAKVSEISGIKKILGVPIFDRPHI
SVKDLSSLNSPSASVRNPSDVELVENNHKIRVLDINLPCDDAVLELDEQAVIEIVVSNKG
SPSKDANSRNHIDLNLSVSEDEEIMTTVPTTDVKVKADIDLEAHAVPESEEDGIHEEKQL
ETSSVSPVGPQDTVEQPQDELMWHAAEAVVIMSSLCFNQVDDATDSPSESPMIDPLN
WFVDVVSSCVNLERKLDNSREKYGMVNDESSPDGLDSFESITLKLTETKEEDYMPKPL
VPENFRVEESGTTSLPTRTRKGPVRRGRQRRDFQKDILPGLTSLSRLEVTEDLQTFGV
LMRATGHSWNSGLTRRSSSRNGCGRGRRRLQVTPSPPPPVATIETSTPLVQQLNNIE
VGGWGKTPRRPRRQRCPAGNPPSIRKSNHT.

In some embodiments, the individual protein comprises a primary amino acid sequence that is conserved by 50% or more relative to SEQ ID NO: 20.

According to some embodiments, SEQ ID NO: 20 consists of the primary amino acid sequence:

MMVDPERVIVDISSDEEVGFVKTRGGGGGSGGSDDYNWITELLGEGDGHSKDDSDD
VVVVREVIMNPKQNLKSLHNSTKPSVNNDDVDDDCVVLEEDPDKPVEVVSDRGDDDD
LLVVSEKGQIACRDYPHSRHLCAKFPFGSTPHERHCDQCHCYVCDTLAPCVYWRTAH
CHATDKDKFWKAERQSTRKSDKALPVVTPGRGNSVSGPPPVANQAPGFIPRLPNYPQ
HNQTFRQTSIRPCSMSSSPGLPNSTRQLSTALRDKFHSHVVSQQLRNASSNVNPGDG
RHVGPQFFTTRTAFKRAGSSRQAIATDRFGYNSSSTCSVPQFGRTHSSTARWQVPW
GSRPVGSNSYIASSQYNTGIRGASAVSFQARLQRQPDLVGVSANYAPMGPQIPSQSN
AGMVGPNAVSSQHQLPRQPSFVGASVNFAPMEPVIPSQISAGIVGANAVSGQPQLPR
QPSHVGVSANFALMEPEMPSHASTGLSGASAVPFQPRLPRHPRFVSASANYAPMEP
RIPSQPQVSVGANSTSSESPTFVLPYAISLSSQSQVGNIYDNPLPPEAQMLSQQYAEG
NSANSLPQQTSVAFHTEGGNTSVNTTASQYLLACSANSGTAPGNSSTAVFQLFNQPD
GETSSNSSVPCNVDLPSPATMGSTFGSRGENTPSGQPELCRLHIPSSSDNQNISQHG
YQGEDTLVPTFEDFDFDWETPISQTNSLGNVFNPKSAEPPEVSRHPQLMESITCVDIQ
YDDWLVSGPSDTPVSTGFNILSPEAPPIDSGFFVDF.

In some embodiments, the individual protein comprises a primary amino acid sequence that is conserved by 50% or more relative to SEQ ID NO: 21.

According to some embodiments, SEQ ID NO: 21 consists of the primary amino acid sequence:

MAASPGIAEPPESSDTHSSPHPPSSPELHPTQDNPSLTTVAPSEMPNPATPVPPPPAE
ESTVEHSEADLLYPTPINEVEQPLSHPPQQAAPSASSAFEASPTEKLASPAPHPEEAAL
EVSHEVTASAPSPSSSPSPQIVGDSLEDAPQPSPPPCTPPATAAHTSTDSSSIEEIAMA
YPCQEEAVRPSPMPESMDAYSRTPTAPLMPPVESRPKGLLPQQQQQPCPPSPEMAF
TPCESLEPVQTLPLQPCHPVECTDASPDATVDEVVEGKLEIAASSLPVPEAGDGHKDIT
SLVLPALDIGSEEMLSQQQRQPLCPEMVPGRGENSKYAHPPQPPPLPESTRGWSNAL
TNEASAVASEEATVALQFGAERSSQEPVQTPMTSRMEPEPCSPETAPPGFEDFKSQ
WMPLPPPIPPAESAHNVARVAASSPVGVMCDVATESLPALEAMGVEMDTPPGLLSPL
KSGAEGRSQQPLLRSCSPLVEAAPCSPDMPPPGFENCKSSWLPLPTIPPVVETTYALL
DVASANTVAFMEKASFVSALESTDVETDTEQCRLSPLEGGTASSLQGPLPISPSPKMQ
SAPCSPDTSPPGFKKAIYVTSKEAPQSSSLDAPPSGSETGKSLPLEHTLVPSHVAEHTV
CALGMVPSGSENVESSQLPQLPAVDLTLDETPDALVDAGTKTVTTEDACNPKPVTGG
KEEANGSMLRPALENDGEDPLPHLEQHASSDIAPTSAEIAPTSFENSESSPQTSPCLAE
TDPSAQASATMLEIVKSDKTSLPLSPLQATGTDMESATLQHSPLKNEESSLAQSEQHP
SSTCSCSPEVAPPGFENLESSEQLPPPPPLSAKFVVPVHFDSTIMHAPAKVISLLNYDY
A.

In some embodiments, the individual protein comprises a primary amino acid sequence that is conserved by 50% or more relative to SEQ ID NO: 22.

According to some embodiments, SEQ ID NO: 22 consists of the primary amino acid sequence:

MWCSSCCALPLLRTFLPSSDGCRLAGPMVPKMVTCLGKPFDIGGLNYDFIEYPLGDTF
IQLNMDQKNFKVSVMTSNVRHLHDHQKLETFGNLKNSFDCYGKYSHGNTEIEKQEHE
SDPEINSYEAVSLTEMEIKCLNYDYVSSSDCITTDANNSKQLCSAVSTSRSSTVGVGIY
KKGGAEGTSGEMVCNKVVPSSTCQNRFAGNECSNDGETSAHFIGGIDSVQQICINSGI
KLETGQPLKKEVNELDLGRRKGNLLNADEDVLKSIVQATFHDKQLKTESSSSNSSHVF
LPASSNDSCQEPSLDVKSCKHSKSSGMNSFCTPFKCSLEESAGKTMVLGTKAQVASR
EVLDSSIFLPRKQGNVGKASETVGNVSERGGEKVLGMIADLMVQRIDVPNSSLLKTKN
EELASRGKLELPCEVDDGLEVASGVARETEPEVSIYREASGSSFSTEEKTGDIVNLNSI
DSSYSDKNDSLIETEPRSKSYNVQVKFENLYSTKVMGEDPKLSMKNQQLCIEVKEQSQ
DRGLGTCQANDGLCGRGSSPLTTPKDVVGGGFDLNEDILADEVEYPKQLVNETSSSC
YVVNVSAPIPVVAKSRVPLCLPMPPLQFEGQLCWKGSAATSAFRPASVSHSPNKRKAL
SNSDDNHSSRHSQGLKGFDLNVAAEESSLEVSPKRAERPNLDLNCLSEDDNCEASPL
VSLPRNSIRDIDLNHNQWFEDTCEDAQDSGQGSQLLRGSAMDPAVSCTGNVRQPGA
SIARPAQPAYRADLSSKQGFSHGPAQTFLVAAPGVIPAHPFPYNKGFYFDPTNPLATIC
HTGVVPCMTDPHGTAVIPHALVSSTPPAFPMAPHLVNVAGGPGPCDVAIIRHSLDLNG
GVGSENGSRGGNAAQLFVPVGNSLVQEQMKSFQQFALPATPIKRREPDGGWDCHQL
GYRQLAAVVQSFNLIPVFAWSQLQLPQTSCDCNLRPD.

In some embodiments, the individual protein comprises a primary amino acid sequence that is conserved by 50% or more relative to SEQ ID NO: 23.

According to some embodiments, SEQ ID NO: 23 consists of the primary amino acid sequence:

MSLPANGTGFCMGTVHKMTSHLCKLRNQVDDICLQVSSDSNTKGPSLSVEDKSYPAN
AFLPSVNVLAFLLFLVASSYFLLRKSVDTFGSCREKHAQKVSSSDSTVSNASMKNDAT
AAYDNLSISESSGCSHRISYMNSYKCDAYFEISDLDKACYMNSLLSLEDDDDSEWLSD
LMPCGSSSEEPSTPLSYKSDAYYEISDFEQGCYIHSLLNLDDEDSEWLSDSKSFERSS
EEPSTPLSYKSDSYYAILDFDKESYLHSLLSLEDEESEWLSDSKSFGCSSDNPSTPFSY
KFDVASEISDLDKAVYMRSLLNFEDEDSECLSDSKSYECSSENPSTPFSCKIDLASEIS
DLDKARYLPSLFSFEDDDSEWLPESKSYACSSENLSTPLRHKSNAFSEISDLDKESYTH
SLINLEDEDSEWFSNSKSFEYSFENPSTPLRYEGQISHLDKAGYFHSLLSLEDDDDDD
DSEWLSDSKPYACSPENLSTPLSYKTDAFSEISDLDKASYMHSLLNLEGEESEWLSDS
KSFGCSSSENPSTPFSYKFDDVSDISDLDKEYCMHSLLILEDENSDRLLDCKSYYECSS
ENSTPLSSKCDSYYNVSDLDKASYLHSLLILEDEHSEWLSDSTSWDIVVPPSVSTCSNF
GDSEDKNLLETAGLEENFSADEPLFWPLEGKVNWNSEETWSSFCTSPRRRFALNSGV
CSVNKKEYFALGEEVPIETLMGLKEFDGHEGLLLDSEFNDVFVVDE.

In some embodiments, the individual protein comprises a primary amino acid sequence that is conserved by 50% or more relative to SEQ ID NO: 24.

According to some embodiments, SEQ ID NO: 24 consists of the primary amino acid sequence:

MVWACEERRNVDAPVRRCERLDTVGTRRGRGWPKCIGESTSLLDVIKEKTFTSPDNE
ADPLPNAGRVALSKKYRTARNPNSDTPKTSEYAFFKKLKKNIGPSNPDLLHRGSKLPKI
TQPSNCISDTTSIFNVLKYPNKDLKIPIQAEKKYKSNECLSFSPVDGVIVHQNAGISKETK
LKYFSLPTAVTPVDSDLQLSPLAGPSNHSVNGHLIRLRRKHKHLFRDIYILSFLGTQYSG
NGIFAAKRQKLCQRANELFLNVEKLNSERFDLVSALLKRLLPGRKEDEGFWDSQVRKG
ENASTSSLAYAKPDHANSRPLSRHIEDVTAPEYRMSEDDCCPSNFFGWPEERVTPEL
DNFTSSFHPIGFDPEAGLHYEDADNSQISNNKITINLWDQSDSSPSLSFERHGLADHFL
PDRLHSAYVPVSRGAQNYFLDWDFNEEDNDPVLAISTIRGNKLYSPIATSRQVDYQQS
TEKKLDALALCSSSLFTNSEYSDVQPYFCSTSFQQKFFPTDVCSKDFGMILEHEDYAV
ARMDRFDLPLLCYSEEQGLLEYCNTEDPFASGTAIVPYLSHHEKNHCDSDALLPMALD
TFGWKFLSATSSPLQRGLSTYQSLRLPHREDTIGLTQEEIKNSLYSLNPRELAPQSVDH
ALNTDIWFSCNSEVSCDKAGGRSLLLANASWITSVEEISPDHCDEWTWS.

In some embodiments, the individual protein comprises a primary amino acid sequence that is conserved by 50% or more relative to SEQ ID NO: 25.

According to some embodiments, SEQ ID NO: 25 consists of the primary amino acid sequence:

MGTKVQSLPGYYSMRDLNEESSSCGWPLFYGDKALANGQYYQNHLPSAAADVCSAY
DKDVVKQTMLEHEAIFKNQILESRPSKVRRKMFDLHLPADEYIDTDEGEKFSDENISGT
TIPDRCCKDGKGDDVNLFCGNGEKTGSHEGTSRSEQCLRSRNGLADLNEPVQVEEIN
AAACIPPLNHNPYQGAAECSDLSAKQKSRFFGFPTEDLLSPHRAPSNNGYLKNDGSG
KLWISSKETGQGKSSSNPIPQVLKREQSFFSSTTIQDTLGKGSEPTSDYLSNRSKTGL
WREKMVGGLDISQRNNAHSTDKYLESVISSHSPGLFAISPSSDFAKSRSLSAREMASS
SLNQKLMSVPIPPSPFLNASGALSRSSQLHQSNGILGDGWPLNINSKHNNPNFHREAP
VHPRIAEHFNNGSVNYNKRSNLICNMTSGKDINLNVRLSNGLVTQSGLGTVDGEQKHG
EQLAVLPWLRSKTTCKNEAQNSGSGRHVTSGELSFLQVASLSKKDETGKGASENFMN
NVTSDLCSNVNGPCMIEAGESSSKNKILGVPIFGMPLISAKESPSLTSLPVSVPNPSVT
GLVENNRKNLVLDINLPCDADVLEVDMDKQSVTEIIVCKEGFSKMEATSRNQIDLNLSM
SEDETVPTTIPTTNVKMKVVIDLETPAVPETEEDAILEEKQVESSSVSPLGPQVTVEQP
QDEFMRYAAEAIVTMSSLCCNQVDDPASSPSETPIMDPLSWFADVASSCVDDMQRKF
DNSRGKICEGKGRSSSQEMDYFESMTLQLEEIKEEDYMPQPLVPEDFILEETGTTSLPT
RARKGPARRGRQRRDFQRDILPGLSSLSRHEVTEDLQTFGGLMKATGHAWNGLTRR
SSSRNGFGRGRRRSQVPPSPPPPVATIETCTPLMQQLNNVEVGLEDRSLTGWGKTTR
RQRRQRCPAGILPSIRLT.

In some embodiments, the individual protein comprises a primary amino acid sequence that is conserved by 50% or more relative to SEQ ID NO: 26.

According to some embodiments, SEQ ID NO: 26 consists of the primary amino acid sequence:

MIRGLLILKHSCYHCLTCIFVKPCPKILIHQLYKSQGLRTLRNSCYHCLTCKFVKTYPKILI
HQFYDIQGLRTLKHSCYHCLTCKFVKTCPKILIHPFCANLMVYLSVCWECNKHLQSYPH
ILQTIITLAHHLFLDVIWLLILDYAVGDWENKFHRDAVIQVGGSNSYASHSSKGTKSLTNT
AFSVQEDVQSKNDTFQDDVLGNKSTHWRKQIANLSEAQKSVIKFVDDEKSHCKTHGSI
NGAITTQQEQLLDSEPKASIAIINLEEFETRQFGESLEKEVSLQHTDFPGIETRCLKGPC
TGRMENGIHTANILAAESHENLKLNWESKEDFEAEINNELLLGEAFFPSQENLYERISKT
KELQPNEFFLASVLSDCGAFETDGEGIADSIFDCELELDEIEESECPFDDFWTEILDVIHL
EQESRDSYSADTVKVTESEVPLHIQDEDELDLLNYQEEPKDPKIANVDTSIDLDIVEDEI
NLPDEDSLISVDHLLSLSDHCSEVTFVGRTDRLKPNLTSDSTGHPEVAHCYEKSFLPG
RNVSSSAIHRKPYPHQISSKTSCIRVNPEIPTFHPLDLYFTCSHFKNIREEKHGHYYPPAI
YFYPWSKIHHESSMKTAPAMEKKYEHLERYPMLYQPDNSLQHMELLRESQADPSEHV
VYDVVQQWDDMQKLPETYHQQLHYPGFGFPI.

In some embodiments, the individual protein comprises a primary amino acid sequence that is conserved by 50% or more relative to SEQ ID NO: 27.

According to some embodiments, SEQ ID NO: 27 consists of the primary amino acid sequence:

MEEADYEGFSIREYTRNVRSVDIRKCCPFPGEFTGDFIQSLLPSLTVTKFRWWSHELS
SLLTKSPDYPKPVFGPKANAESTVPCKKRSIGETTTNDLVLHTKKIKTKKLYDSFDAKTD
NKVCNCEEQARERAGDDMKCSLGIKERSGVMSPPSMSKAGVRCFSYTEEEELFPDS
SKPVSQDLDSELQIPRTLKVAKRKVCSVRSLDKSSIDASQCETQVRVSLHLCCSQMNR
LSLCSVAEDQSAKVMSNDKTSDNMLLESKQVNKFQPVVSERRLSHALVQSSMSCFSG
SHLTLERIKDALDLERKRHVSQPNQSSISASSENHYRSCSSSLSQPASVAIHRQTLLNQ
SHFNPLLVEEAMSGFPLNLQGELVEANGSFRSAGAENDILLSSFVDLSSGTEPALATDV
GRFTVHSVKHNKYYPARLGLDETFTENAFLVSDTNNPHTINLPKQEALFQNLSNRNMV
TNSDGLCLHDTQSTMRLMGKDVSVKTGYSDVVGRGEIIASDPSMDYSFLGSYAQQSS
WLWRTATLGESHSITSLDKSSKDPFPMFCEPHVGLAPQDRTAVVPDSELPSTLLHPCG
SLVSCAFTDKDLYFHETGLGQQLNSVTFSQQQLPFPSNPEIAGLPSDDKNVGVVGLLP
DVREQSLGLPFSCTDSTRQSLLHWPQSSFESSRLDVSYLHQSR.

In some embodiments, the individual protein comprises a primary amino acid sequence that is conserved by 50% or more relative to SEQ ID NO: 28.

According to some embodiments, SEQ ID NO: 28 consists of the primary amino acid sequence:

MGALSSPTQWILEDDLLLKNAMEFQQAGASLESLAKGAVSFSRRYTLKELYDRWSSIL
YDSNASLETSARILGFENESSTTNPSKTGRTCSSKGKDFLSHKRKVNSVRNHYYAMRK
RICTEPCNTADLDFLVAPCSCAANGRQCICRGSLNSQKLHSVENIAIGVPVVNCYEQSG
TTYNGGQNVFPDGIVDRDCLCRSHVNQSFEHDYIQKDPQICRENHIPLRNSFDVGETD
GLQSLPSTNLYKDEIIEAKQFPICDSQIGKSEGFIVNRSEVPDSGDSFDLLGTIWKGSAP
TMPMDIHITEEDPEGLTRNDTNSLQGKLNDGTADDGMNSTELISENDFIDFTGSYMEFS
ADDEFLFMDVDETPIVSSSPRNANQVDVANASDPKPADAIESTLVAPDLRSEGTNNSS
DPIGSELDIHNATNADMEVPHTGETIEIFLICTLNTEDTEIPCNDDIILPTCESNSKHQSVS
LPPSIKILSVEGKSTVGDLPIVKEENVGNTLPLTLPAKAEPSGLQHKVGLVLPSNGCTGA
EFTGHPINSVNDPNSCTIHTTALKEECVAGDLGQHGNFNKSRDFFLEKPVPQVDHVNY
YSANIADGPKQEIDDQIGPQKCVPANAAGDDLGLQGPIATVSTSDQEEQFSESDDDVP
SYSDVEAMIIGMDLGPYDPESRPITKEGLLGDIVSR.

In some embodiments, the individual protein comprises a primary amino acid sequence that is conserved by 50% or more relative to SEQ ID NO: 29.

According to some embodiments, SEQ ID NO: 29 consists of the primary amino acid sequence:

MAMALALSCGRRQRGKKGGGGGGDCGGRHFHVHYHLPRRVCASSFVPNLL
PRFFSLLSACVLVPPVLFLAVLAFLVLFLWFTLLYFIRSLWNKDINGETF
ERSSNDHGSDADKRLGEGVAEEGKAEKKTLKISSEDCARRFEIEEVLHVN
SDDSQNLPSMVSSDGCLNCKMHTDDEKLIKEVTVFEIKRRESGAAIVCSD
GLSQKRQPRDMSVDWFEEETKSGDIIKSGDSSPTVLSTFSSEFHDFSDNN
EVVGLATDFLDVNNQNKAVLLDSSSHRGIMDNHCKCGEEFSSDKEAPAYH
LFENYDFVDKHETKEVVLGDTVVLTFVDDLANDDSEYREDISEQKDPNNL
SGLLDNVADIFYQQQEIHKVVVSDDNKPPEVLFLSGKQTVSSSGEFSSPN
ENGTSGFPFDSVCEDKNGTVAYPSVASHYNIGIEDDKCEDHTDNNIEEAS
SIASTNCGSADKHQTIQVLPVCLANGDNIDDVASLGSSICEDVEDKDNKS
NANCISEGAPHGNGMPLVRSPASWWNLCGVIDVFAGCKD.

In some embodiments, the individual protein comprises a primary amino acid sequence that is conserved by 50% or more relative to SEQ ID NO: 30.

According to some embodiments, SEQ ID NO: 30 consists of the primary amino acid sequence:

MGEEQYVETTPTLQSQPTHTLHSQIVPLSHSQPNVPIENHGVFHSSLTGD
DYVCVGRSVRDMSNNIVLPSSLFREHE.

In some embodiments, the individual protein comprises a primary amino acid sequence that is conserved by 50% or more relative to SEQ ID NO: 31.

According to some embodiments, SEQ ID NO: 31 consists of the primary amino acid sequence:

MIIEPEKMISEPEKTISEPEKMIPEPETSIPESEKMIPEPETTIPESEKM
IPEPETTIPESETTVPKTNLKSICRRPRFIASSSRLSVVVVVVSPSSSPR
PCRRRLSVVVVVVVSPSSSSPLR.

In some embodiments, the individual protein comprises a primary amino acid sequence that is conserved by 50% or more relative to SEQ ID NO: 32.

According to some embodiments, SEQ ID NO: 32 consists of the primary amino acid sequence:

MNQRPRNDYMRVNRTGKYTRKSIFDQVHTNANSIPHQPRQPQLKTQVYII
DKEDFKSIVQQLTSNQSCEFLPQNLPNRQKSRPEPTSPVPLNATGVHVSS
HMGYIESLLEESSDSSGDNFQQSFDENQSHIQPQVYSNGDNFQQTFDKYQ
SHMQPMSYSNGPKPVMTTTLPTPWFNGSPQQIDSAYSLQSTRVEYPQPLT
PNFSFSSVTQPGFFDPDLRRF.

In some embodiments, the individual protein comprises a primary amino acid sequence that is conserved by 50% or more relative to SEQ ID NO: 33.

According to some embodiments, SEQ ID NO: 33 consists of the primary amino acid sequence:

MMMTSGSEVVILPSPKPREATVMCLIRMSFREGPDEEQNCGHQANQESSS
SIQKPDQTQDVGVMQFANQETFSSREFGPYGSSSPHFDPYREGNTWFRRT
WMLAEESL.

In some embodiments, the individual protein comprises a primary amino acid sequence that is conserved by 50% or more relative to SEQ ID NO: 34.

According to some embodiments, SEQ ID NO: 34 consists of the primary amino acid sequence:

MTEYWLASENDAQFQEVVLCHLRDNNKMVVDQPPESKNGDNDIATEQPQQ
GNSDDNNNRLLDFTHQQRPLIPPFEGQGLRLQTIMGYSDKATQEQQHPPI
SPPPQRQDSGSINNALVIMEDECVSQDEIFNLADLEAGITHPQQQHRQMM
VDPYDDISFSRLAMPNNLIYHHEDSWHQDTSPWNNTNPRGLIFNSHGCEI
QDQTVTKGVNQDSYY.

In some embodiments, the individual protein comprises a primary amino acid sequence that is conserved by 50% or more relative to SEQ ID NO: 35.

According to some embodiments, SEQ ID NO: 35 consists of the primary amino acid sequence:

MYTNEWELSEHESFEPFSIVTPLEDITTIIHETNKSFQPQQQYYQQQIEF
PIWSNEFSMETTTESPFQFFQENYSNNIAKQDQPTTSFDFVVESLMSASS
EDSISYSEKCSELASCSDNKPICENSPQQHKKVLEGYSALHQKSREISFQ
KTQLESCINQDKQCSPCGFDFSTSTNCDSKVTTRAKRRLRWTKEMHEPFV
MIVNQLGGPESKFI.

In some embodiments, the individual protein comprises a primary amino acid sequence that is conserved by 50% or more relative to SEQ ID NO: 36.

According to some embodiments, SEQ ID NO: 36 consists of the primary amino acid sequence:

MIEVEENLNVEKVIVSANLENEEVVASPTKREKSKFVIMAPHQAFALVPK
EGQSKQNIDQAFPKSLPHFYQSFPMQNGVGEGIKNGFKENDIVLEEMIET
SVILDNEPKEQMPNWTSPSLLNPRSSCENIFKFVNIMSCHKLNEQNRVDA
EESEDYNEKIMVPKHLVEEFRQFESHDKPNSEEIEIVN.

In some embodiments, the individual protein comprises a primary amino acid sequence that is conserved by 50% or more relative to SEQ ID NO: 37.

According to some embodiments, SEQ ID NO: 37 consists of the primary amino acid sequence:

MYILLQAVFTIATTALTVPIFLSYKLHVVFQIFKVSASIWNGGNFMLDVM
PRQVILKEEK.

In some embodiments, the individual protein comprises a primary amino acid sequence that is conserved by 50% or more relative to SEQ ID NO: 38.

According to some embodiments, SEQ ID NO: 38 consists of the primary amino acid sequence:

MDRHVILFLRCPWRRYVEVGNAEVDGENGVVEQDKYSIRPAHTISETEKN
QITSSWRLS.

In some embodiments, the individual protein comprises a primary amino acid sequence that is conserved by 50% or more relative to SEQ ID NO: 39.

According to some embodiments, SEQ ID NO: 39 consists of the primary amino acid sequence:

MALPHHHLQLHIQQQQPHQQQQQQQSKSYRDLYNNMDGQITTPVVYFNGS
NLPEQSQHPPYIPPFQVVGLAPGTADDGGLDLQWNYGLEPKKKRPKEQDF
MENNNSQISSVDLLQRRSVSTGLGLSLDNGRLASSCDSAFLGLVGDDIER
ELQRQDAEIDRYIKVQVFTPSID.

In some embodiments, the individual protein comprises a primary amino acid sequence that is conserved by 50% or more relative to SEQ ID NO: 40.

According to some embodiments, SEQ ID NO: 40 consists of the primary amino acid sequence:

MWQLEAAARSAIENGMEEGHGDSTDEFVPIAFPQAMHMEMEANNVPVRNN
NPTITALRRYEDQIVPSMSSSFFRNEDEETSVDIRNSVVDSRAEVPIISM.

In some embodiments, the individual protein comprises a primary amino acid sequence that is conserved by 50% or more relative to SEQ ID NO: 41.

According to some embodiments, SEQ ID NO: 41 consists of the primary amino acid sequence:

WSKIAKHLPGRTDNEIKNYWRTRIQKHIKQAENSQQQSSSDIQINDNDNQ
IGSTSQISAMPEPMDINIISPPSYQGMLEPFPNQFPTISDQSSCCTNDNN
YWSMEDLLSLQLLNGD.

In some embodiments, the individual protein comprises a primary amino acid sequence that is conserved by 50% or more relative to SEQ ID NO: 42.

According to some embodiments, SEQ ID NO: 42 consists of the primary amino acid sequence:

MTRAYSVKGKKRKNKDVVEKYHREDKEVADEEQVQPKKPNLQKEEEPTQT
PTPKITEENNNNELVGIPIAPPTENNSEKPGVIFILEKASLEVAKVGKVL
QFSALFLL.

In some embodiments, the individual protein comprises a primary amino acid sequence that is conserved by 50% or more relative to SEQ ID NO: 43.

According to some embodiments, SEQ ID NO: 43 consists of the primary amino acid sequence:

MTNETTETTTMVTSQQVVEVSEPSKKPIIPFFPNFNFNFQIPQFPFPQFL
PKNHRHDDAGGADKNNETPQLHSEGGQPGPNVVTFPKSQQVVVPSPLQAE
ADANSSTAKTSHPIVIYQVYAIGAFFLSQWIWARWNERKARGGSPDDEGR
GPQDNK.

In some embodiments, the food ingredient comprises a salt. In some embodiments, the salt comprises ions of sodium, magnesium, calcium, zinc, iron, manganese, phosphorous (e.g., phosphate), chloride, sulfate, or combinations thereof. In some embodiments, the salt comprises a chloride salt of sodium, magnesium, calcium, zinc, iron, or manganese. In some embodiments, the salt comprises a sulfate salt of sodium, magnesium, calcium, zinc, iron, or manganese. In some embodiments, the salt comprises a phosphate salt of sodium, magnesium, calcium, zinc, iron, or manganese.

In some embodiments, the food ingredient comprises a fat. Fats include but are not limited to: saturated and unsaturated fatty acids, oleic acid, lauric acid, palmitic acid, linoleic acid, and alpha-linoleic acid, coconut milk, coconut oil, palm kernel oil, palm oil, laurel oil, pecan oil, canola oil, peanut oil, macadamia oil, sunflower oil, grape seed oil, sesame oil, sea buckthorn oil, karuka oil, nutmeg oil, soybean oil, cocoa butter, olive oil, salicornia oil, safflower oil, primrose oil, melon seed oil, artichoke oil, hemp oil, wheat germ oil, cottonseed oil, corn oil, walnut oil, rice bran oil, argan oil, pistachio oil, flax oil, hemp seed oil, lingonberry oil, and camelina oil.

In some embodiments, the food additive comprises a sugar or a fiber. Sugars and fibers include, but are not limited to: galactose, glucose, sucrose, lactose, maltose, ribose, trehalose, fructose, wheat starch, corn starch, potato starch, mung bean starch, arrowroot starch, kuzu root starch, cassava starch, tapioca starch, pectins, chitins, xanthan gum, psyllium seed husks, chicory fiber, (malto-)dextrins, and glucans from plant and fungal sources, agave nectar, brown rice syrup, corn syrup, coconut sugar, maple syrup, malt sugar, sugarbeet, moonrot, Selaginella, algae, shitake mushrooms, oyster mushrooms, and golden needle mushrooms.

In some embodiments, the food ingredient comprises a trace nutrient. Trace nutrients include, but are not limited to: L-cysteine, selenium, folate, riboflavin, vitamin E, vitamin A, vitamin C (ascorbic acid), vitamin B12, vitamin B1, vitamin B2, vitamin B3, vitamin B5, and vitamin B6.

### EXAMPLES

The following examples are included for illustrative purposes only and are not intended to limit the scope of the disclosure. Steps such as “computer processing” are carried out by a computing device comprising at least one processor and at least one non-transitory computer-readable storage medium (i.e., “memory”) comprising processor-executable instructions, wherein the at least one processor is configured to execute the processor-executable instructions to perform the computer processing.

### Example 1: Generation of a Candidate Protein Database

Publicly available plant, fungal and protist total proteomes were collected, annotated, and analyzed to build an in-house database of candidate protein sequences (see FIG. 2). All incomplete and redundant sequences were removed, and all non-standard amino acids were converted to standard ones. The disordered regions (e.g., IDRs and/or disordered regions of IDPs) of each protein in every proteome were determined, allowing for the annotation of all folded and disordered domains of each protein in every proteome.

This candidate protein database was then further curated by clustering and filtering similar protein sequences. Proteins were clustered at a 90% sequence similarity threshold using the computer program “CD-HIT” (for a description of CD-HIT, see Fu et al., “CD-HIT: accelerated for clustering the next-generation sequencing data”, Bioinformatics 2021).

In order to convert primary amino acid sequences into machine-readable, fixed-size numerical vector representations, numeric sequence embeddings were computed for every candidate sequence using a protein Large Language Model (pLLM) encoder pre-trained on the UniRef50 database.

### Example 2: Generation of Target Protein Datasets

A target protein dataset was compiled by extracting all mammalian alpha-, beta- and kappa-casein homologs, respectively, from the Uniprot, InterProt and NCBI databases. Another unrelated target protein dataset was compiled by extracting all wheat, rye, and barely gliadin and glutenin proteins from the InterPort and GlutPro databases. These target protein datasets were then curated by removing incomplete and redundant sequences and converting sequences with non-standard amino acids to standard ones. Redundant sequences were removed at a 90% similarity threshold using CD-HIT, as in Example 1. Further, this curation removed any subsequences that are not part of the mature proteins in nature, in particular signal sequences. The curation was conducted by at least computer processing the target protein datasets. The computer processing comprised use of a trained computer model, wherein the trained computer model comprised a bidirectional recurrent neural network (RNN) with long short-term memory (LSTM) architecture (for a description of a RNN with LSTM, see Griffith and Holehouse, “PARROT is a flexible recurrent neural network framework for analysis of large protein datasets”, eLife 2021, which is incorporated by reference herein) on a dataset of eukaryotic and prokaryotic signal sequences (for a description of signal sequences see Rapoport, “Protein translocation across the eukaryotic endoplasmic reticulum and bacterial plasma membranes”, Nature 2007, which is incorporated by reference herein). We then computed protein embeddings for every target protein sequence in our database using a pLLM, as described in Example 1.

### Example 3: Extraction of Physicochemical Properties and Sequence Motifs

For every primary amino acid sequence in each target protein dataset, the physicochemical properties were then determined. The physicochemical properties were determined by at least computer processing every primary amino acid sequence in each target protein dataset. The computer processing was conducted using custom computer programs.

The determined physicochemical properties included but were not limited to: isoelectric point, hydrophobicity, charge clustering, the fraction of charged residues at various pH values, the net charge per residue, and the presence of disorder (e.g., intrinsic disorder). The conservation of amino acid composition was also determined to identify evolutionarily conserved sequence motifs. To identify said conserved sequence motifs, all possible dipeptide combinations across all sequences in a given target protein dataset were determined, and the observed frequencies of these dipeptide motifs were compared to their expected frequencies. All dipeptide motifs determined to be abundant and statistically significant were extracted. The frequencies, spacing and clustering of these dipeptide motifs (e.g., the presence or number, the distribution, normalized sequence asymmetry parameters) were then determined. The values and metrics provided by these determinations (e.g., physicochemical properties) were utilized as part of a heuristic model to predict target protein behavior (e.g., target functionality, target food functions) in unrelated proteins (e.g., candidate proteins, individual proteins).

### Example 4: A Trained Classifier Model for Use in Predicting Target Protein Function in Unrelated Proteins

A trained computer model was developed for use in predicting target protein behavior (e.g., target functionality, target food functions) in unrelated proteins (e.g., candidate proteins, individual proteins). The trained computer model comprised a machine learning (ML) model, wherein the ML model comprised a bidirectional RNN with LSTM architecture. The trained computer model was trained using the three casein target protein datasets from Example 2 and a control dataset of random disordered proteins. Using the trained computer model, each candidate protein was classified as an IDP or as a protein comprising an IDR with similarity to casein were classified as either a random IDR or alpha-/beta-/kappa-casein.

Next, complex non-linear patterns in the classified protein sequences were determined as in Example 2 and used to develop a trained computer model for further evaluating predicted target protein behavior (e.g., target functionality, target food functions). Using this model, each protein classified as an IDP or as a protein comprising an IDR with similarity to casein was evaluated for similar physicochemical properties and sequence motifs as in casein. Similarity was determined based upon a protein having physicochemical properties and sequence motifs within one standard deviation of all physicochemical properties and sequence motifs determined in casein. The highest scoring candidate proteins classified as alpha-/beta-/kappa-casein were selected for experimental validation.

### Example 5: Experimental Validation of Target Protein Behavior in Unrelated Proteins

Genes encoding SEQ ID NO. 1 to 43 were synthesized and cloned into custom bacterial expression vectors comprising the tags detailed in SEQ ID NO. 44, 45 and 46. The corresponding proteins to the expression vectors were expressed in Escherichia coli BL21, or derivatives thereof, for 4 hours at 37° C. The cells were harvested by centrifugation and resuspended in 50 mM Tris pH8.0, 7M GHCI and 2 mM TCEP. The cells were lysed by addition of an equal volume of 200 mM NaOH and 1% Triton X-100, followed by gentle rocking for 5 min. The crude lysate was neutralized by addition of 100-150 mM acetate buffer pH 4.5. The neutralized lysate was cleared by centrifugation at 20,000×g for 1 hour at 20° C. The proteins were purified from the cleared lysate by immobilized metal affinity chromatography. During washing, the buffer was exchanged to LSB (44 mM Tris pH8 and 290 mM NaCl) and the proteins were eluted off the columns in LSB supplemented with 400 mM imidazole.

Analytical assays were conducted to validate the presence of the predicted target food functionality. Phase separation of the proteins from aqueous solution into a light phase and a dense phase at low pH (a target food function present in casein) was evaluated using qualitative and quantitative assays. For qualitative phase separation tests, 200 mM acetate buffer pH4.5 was added to the proteins in solution and the resulting mixtures were incubated for 1 minute. A turbid solution following addition of the acetate buffer indicated phase transition, as shown exemplary for casein replacement candidate 5 (CRC5) in FIG. 12A. Quantitative analysis comprised cleavage and removal of the tags, and further purification of the proteins by gel filtration and reverse IMAC. The purified proteins were then evaluated for pH-triggered phase separation (using 200 mM acetate buffer pH4.5) at various protein concentrations, using the protein concentration in the light phase as the quantitative measure of the target functionality, as shown exemplary for CRC5, CRC10 and CRC19 in FIG. 12B. Protein concentration was determined by measuring protein absorbance at 280 nm using an Eppendorf photospectrometer. The data was analyzed by fitting the measurements to a one-phase exponential function to determine the saturation concentrations at which CRC phase separation occurs. Rheological measurements were performed with a Piuma nanoindenter to gain quantitative insights into the material properties of phase separated CRCs, as exemplified for CRC10 in FIG. 13.

### Example 6: Example Food Compositions Made from Predicted and Validated Candidate Proteins According to the Invention

A cheese matrix comprised of 45% recombinant CRC5, 52% canola oil and 3% CaCl2 was formed by first preparing an emulsion of all ingredients through vigorous stirring at 2000 rpm at 50° C. for 30 minutes and then adding 100 mM acetate buffer pH4.5 and further incubation for 30 minutes at 30° C. to induce curdling. The curdled solution was transferred to 4ºC and incubated for at least 16 hours. The curds were then separated from the liquid material, shaped into form, and dried at 50° C. to yield a cheese matrix (FIG. 14A).

In another embodiment, a cheese matrix composed of 65% recombinant candidate protein mixture, 32% canola oil and 3% CaCl2 (Table 1) was formed as described above. The curdled solution was then incubated for 16 hours at 4° C. The curds were separated from the liquid material and dried as above. A photograph of an illustrative example is shown in FIG. 14B.

TABLE 1

| Ingredient | % |
|---|---|
| CRC10 | 36.5 |
| CRC12 | 28.5 |
| CaCl2 | 3 |
| Canola Oil | 32 |
| Total | 100 |

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations, or equivalents. It is intended that the following claims define the scope of the disclosure and that methods and structures within the scope of these claims and their equivalents be covered thereby.

While the above description provides examples of systems and methods, it will be appreciated that other apparatus, methods, or systems may be within the scope of the claims as interpreted by one of skill in the art.

1. A method of predicting at least one target food function of a candidate protein, the method comprising:
(a) providing an amino acid sequence for the candidate protein;
(b) computer processing, by at least one processor executing a trained computer model, the amino acid sequence of the candidate protein to predict a set of candidate amino acid sequences having intrinsic disorder,
wherein the intrinsic disorder comprises a lack of stable secondary structure along at least about 10% of the length of an individual candidate amino acid sequence; and
(c) computer processing, by at least one processor executing a trained computer model, the set of candidate amino acid sequences to generate an output that is indicative of the predicted target food function or target food functions of the candidate protein.

2. The method of claim 1, further comprising computer processing the set of candidate amino acid sequences to determine a set of physicochemical properties of individual candidate amino acid sequences.

3. The method of claim 2, further comprising computer processing of the set of physicochemical properties to generate the output.

4. The method of claim 2, wherein the set of physicochemical properties comprises one or more of:
sequence length,
molecular weight,
isoelectric point,
pH-adjusted net charge per residue,
a presence or number of hydrophobic amino acids within the sequence,
a presence or number of aliphatic amino acids within the sequence,
a presence or number of aromatic amino acids within the sequence,
a presence or number of positively charged amino acids within the sequence,
a presence or number of negatively charged amino acids within the sequence,
a presence or number of polar amino acids within the sequence,
a presence or number of glycine amino acids within the sequence,
a presence or number of alanine amino acids within the sequence,
a presence or number of cysteine amino acids within the sequence,
a presence or number of proline amino acids within the sequence,
a presence or number of histidine amino acids within the sequence,
a distribution of hydrophobic amino acids within the sequence,
a distribution of aliphatic amino acids within the sequence,
a distribution of aromatic amino acids within the sequence,
a distribution of positively charged amino acids within the sequence,
a distribution of negatively charged amino acids within the sequence,
a distribution of polar amino acids within the sequence,
a distribution of glycine amino acids within the sequence,
a distribution of alanine amino acids within the sequence,
a distribution of cysteine amino acids within the sequence,
a distribution of proline amino acids within the sequence, and
a distribution of histidine amino acids within the sequence;
wherein the distribution is expressed in terms of the mean inverse distance weight parameter of amino acids within the sequence.

5. (canceled)

6. The method of claim 1, wherein the trained computer model is trained using a training data set comprising:
a set of amino acid sequences of individual proteins with the target food function;
and/or a set of amino acid sequences of individual proteins without the target food function.

7. The method of claim 6, wherein the trained computer model is trained using a training data set comprising:
a set of amino acid embeddings of individual proteins with the target food function derived from a protein large language model (pLLM); and/or a set of amino acid sequences of individual proteins without the target food function derived from a pLLM.

8. The method of claim 6, wherein the trained computer model is trained using a training data set comprising:
a set of physicochemical properties of a set of amino acid sequences of individual proteins with the target food function; and/or
a set of physicochemical properties of a set of amino acid sequences of individual proteins without the target food function.

9. (canceled)

10. (canceled)

11. (canceled)

12. The method of claim 1, wherein the set of physicochemical properties comprises:
a presence or number of clustering motifs within the sequence; and
a distribution of clustering motifs within the sequence;
wherein the distribution is expressed in terms of the mean inverse distance weight parameter of clustering motifs within the sequence.

13. (canceled)

14. The method of claim 1, further comprising:
recombinantly expressing one or more candidate amino acid sequences within the set of candidate amino acid sequences to provide quantities of candidate proteins.

15. The method of claim 14, further comprising:
conducting analytical assays to validate the target food function for each of the candidate proteins.

16. (canceled)

17. The method of claim 15, further comprising:
selecting one or more of the candidate proteins as potential food ingredients if the individual candidate proteins are determined to have the target food function satisfying a predetermined criterion.

18. The method of claim 17, further comprising:
assessing the one or more candidate proteins selected as potential food ingredients to determine whether the candidate proteins meet desired performance requirements as part of a food preparation.

19. (canceled)

20. (canceled)

21. (canceled)

22. The method of claim 1, wherein the target food function comprises phase separation of the candidate protein or a domain thereof from an aqueous solution upon exposure to one or more environmental triggers to form a dense phase and a light phase, wherein the target food function is inversely proportional to the concentration of the candidate protein or a domain thereof in the light phase.

23. (canceled)

24. (canceled)

25. (canceled)

26. (canceled)

27. The method of claim 22 wherein the one or more environmental triggers comprises one or more enzymes.

28. (canceled)

29. (canceled)

30. (canceled)

31. (canceled)

32. (canceled)

33. (canceled)

34. The method of claim 1, wherein at least one analytical assay determines the concentration of the candidate protein in aqueous solution as a variable function of one or more environmental conditions.

35. (canceled)

36. (canceled)

37. (canceled)

38. (canceled)

39. (canceled)

40. (canceled)

41. (canceled)

42. (canceled)

43. A composition comprising a candidate protein predicted to have the target food function according to the method of claim 1 and one or more food ingredients.

44. A food product comprising the composition of claim 43.

45. A food product comprising a candidate protein predicted to have the target food function according to the method of claim 1.

46. A composition comprising:
an individual protein, wherein the individual protein comprises a primary amino acid sequence that is conserved by 50% or more relative to any one of SEQ ID NOs: 1 to 43; and one or more food ingredients.

47. A food product comprising the composition of claim 46.


SEQUENCE LISTING (49 sequences)

SEQ ID NO: 1 - protein, 59 aa, Lupinus albus
MEFQQAAVEANKPVQHQQQQIMDYFSFFEEDQPASTLLQLAAPVLPHLQLAQPNIQDSP

SEQ ID NO: 2 - protein, 76 aa, Lupinus albus
MPLNNNTPSYSHPRNSNIIFNFILHLLTTTSSSILSKSQNLSQLVDQENKDVDLVVSSSFVNVEELSLIQSDSKPK

SEQ ID NO: 3 - protein, 62 aa, Lupinus albus
MSTELVSIGQNQVPNISNIGESNSVLTNVDAPPIQDETHFSSPTDVEEARPKQISSIWNHFE

SEQ ID NO: 4 - protein, 114 aa, Artemisia annua
MSNENEYVDEKKVKPLACFSSPIVVSSSYPQHSLFEAPSFSLGPEFEDDPETEDEKPVIKEICIRQNPRRDVGVAAHSRSPFKFRQILPNVPIKPSEERVSDILFMIPEETNQV

SEQ ID NO: 5 - protein, 100 aa, Artemisia annua
MNNQPPSPNNVLDNFILPSSDTNSLIANYGSEEIVNNNVVPQAPTKSVGMGNGWFMQNNQQQYPMINLEELMQDDVWSNPQNDQVTTSPFRGIGDEEVWE

SEQ ID NO: 6 - protein, 132 aa, Artemisia annua
MVFPLPDERNWHNPGNLPKLKPPAMVKQQSGRPKNKDRFPSKYEEPISKQCGRCGCKSHTREACMQPLPKSGNGKQPFDCSGVRKRKSDIEREVIIESNPGNETEVINNPNMEVDEEYFENGRIYADWEDLD

SEQ ID NO: 7 - protein, 64 aa, Solanum sp.
MYWILNNLVWNRVDPIVEMGLGQPDSFVEEGEPQSYTIPLRFHSEKSSFRKNQIQELSPEIRRN

SEQ ID NO: 8 - protein, 70 aa, Solanum sp.
MDQNNNQEMYSHSIQPTQITQQIILPPLTTTIIQQIFHPPFPTTITCMPTTTAIPQEVIDLQQQPKKLDD

SEQ ID NO: 9 - protein, 136 aa, Solanum sp.
MHICKTSQSSIFISYENQKPNQCQIKKQKVGKLNTIKEETVPFTPLKCESQNAQFFVNSIEPFTSEYMYLPLLPNQEEEFEMDKIFQSSELDRIEFSSMPTSIIESPFCREWDLSVYDQLDVHDPNSMINISEYFT

SEQ ID NO: 10 - protein, 140 aa, Gossypium sp.
MGQEQNQNHHRQQAEQPQQPPLSVGISPEQHPLHPDDGYPLESRDSSNHKRPSNNTSYEEFVAAEEERQRLILSEDMDSNGFPNSWPKVSPPPFLPVTVAPPTVEPPSPEKDFSPPTVTRNIESPPLRPPPPPSKCCIIL

SEQ ID NO: 11 - protein, 133 aa, Gossypium sp.
MADANNKEAQSKSKNPLEPRNFFSMLPRVEFKFPSFDQQSEKPDASVEKEEQIEIAKPPSVFMGNRRKNPPPLEFEAEECLGRTSNPIVLWQVSQPYCHSAEPYMRLSFNSFEVIGFFPRSRIFSPLPLCPEE

SEQ ID NO: 12 - protein, 78 aa, Gossypium sp.
MLEEENNNMYRWIQEHRAAIEYQQHGGLEAKPVEHHQQVLDEFPFYGEPSSVLQLATIPQQFSYQLQLAQPNLQDSNV

SEQ ID NO: 13 - protein, 72 aa, Ensete ventricosum
MELQNDNMYLRNKVAENERVQQQMDMLPSTATTTEYEVMPSYDSRNFLQVSIMQPNQHYTHQQRTALQLGRV

SEQ ID NO: 14 - protein, 111 aa, Ensete ventricosum
MGGRKCHAASQEERECFEAHHLRRFNSSSMTQRDHNLKELMSIPFPFNLPDEVKDLRLQLPCPPPFIPRHSPHDFFTSSSEGEWWNQVRCCTAFEIASQEGNGKQQAEHDR

SEQ ID NO: 15 - protein, 75 aa, Ensete ventricosum
MELQNDNVFLRNKITENERTQQQMNILPSTSEYDILTPFDSRNFLQVLQPAQHYSQQQQQAGLQLGYGQLVMNTN

SEQ ID NO: 16 - protein, 67 aa, Vitis vinifera
MEQQHVEPQIPVDLDGKVEDGRQQLVENDQQPIVAQDEQHPKCAIRKSIWMTNYERLSKSKMEEGNG

SEQ ID NO: 17 - protein, 130 aa, Vitis vinifera
MPETEEVIEEGRDNTIEPSPPSKQFGFEDVFTEIPNQSLSAEGVLNLEPDPFMKQLPHRHNRGIPKPTYEPELSTKVKYPMSNYVSNHRLSESNKSFVNQLSIVAIPNSVQEALADPRWKTVMNEGMKSL

SEQ ID NO: 18 - protein, 146 aa, Vitis vinifera
MDVMFHEDSIYFSSESELQGEYHNEIQTFDYDYHISKEDEFGQSELVNQEVGELDMSGQQFGSEDVFTEIPNQLSSVEGVLNLEPDPFMKRLPHCHNRGIPKPTYEPDLSTKVKYPMSNYVFNHRLSESNKSFVNQLSTVIIPNSV

SEQ ID NO: 19 - protein, 1077 aa, unidentified
MGTKVRNLPEYCSIRDHNEESSSCGWPLFYGDKTVTNGQYCHNYLSSATADACSVYDKDILKQKMLEHEVTFKNQVFELHRLYRIQRNLMGEVNMKELHRNKIPVETSFTTGPLASQITSEDGKKWYTHSFPIGSSTCARPSISGFEGSHSPLDTNKGISKQAGKFPSPKESSSKDVEVLKCRPSKARRRIFDLHLPADDYIDTQESEKLGDERKSGATLFLPDRNCNHDVNVFHSNGGKTGSQQDSSRPEKSLKSRNGLADLNEPIEIEEINDASYVPLLNHNLYLGKTECSDLCAKQNSRFFGLSTDDLINSHHGTDSWARNNGYMENDGSGKGFVLSGLETGLAKSNLKPIPHVFKQEQSLLSPQNMKDTLSMAHKPTSDCRTNQSKADLWWEKAVSSLDVSEGNHEYSTNKYAESVVSSHRSSLFAIAPSPDLVKSWSHSPSSLEMPTSSLNQKTASAQTPPWLNASGVLSRSSQSHQSNAILGSTWPQHINSKANPGFQCEVPLQNGFYPGSSSGCKELSANISSISYDYLNHNNNDRKRIPERCSNDSAKYYESSNSNCNNKQSGKDINLNVLLSNGSSNMLVTQSGPGIMEGEQKHEEQIAVLPWLRAKTACKNEMQNVARGLTTRELGFSHVTSLSDKDEIGQGSSEKVMHNVTSGLCSNDNEPRRAKVSEISGIKKILGVPIFDRPHISVKDLSSLNSPSASVRNPSDVELVENNHKIRVLDINLPCDDAVLELDEQAVIEIVVSNKGSPSKDANSRNHIDLNLSVSEDEEIMTTVPTTDVKVKADIDLEAHAVPESEEDGIHEEKQLETSSVSPVGPQDTVEQPQDELMWHAAEAVVIMSSLCFNQVDDATDSPSESPMIDPLNWFVDVVSSCVNLERKLDNSREKYGMVNDESSPDGLDSFESITLKLTETKEEDYMPKPLVPENFRVEESGTTSLPTRTRKGPVRRGRQRRDFQKDILPGLTSLSRLEVTEDLQTFGVLMRATGHSWNSGLTRRSSSRNGCGRGRRRLQVTPSPPPPVATIETSTPLVQQLNNIEVGGWGKTPRRPRRQRCPAGNPPSIRKSNHT

SEQ ID NO: 20 - protein, 776 aa, Capsicum chinense
MMVDPERVIVDISSDEEVGFVKTRGGGGGSGGSDDYNWITELLGEGDGHSKDDSDDVVVVREVIMNPKQNLKSLHNSTKPSVNNDDVDDDCVVLEEDPDKPVEVVSDRGDDDDLLVVSEKGQIACRDYPHSRHLCAKFPFGSTPHERHCDQCHCYVCDTLAPCVYWRTAHCHATDKDKFWKAERQSTRKSDKALPVVTPGRGNSVSGPPPVANQAPGFIPRLPNYPQHNQTFRQTSIRPCSMSSSPGLPNSTRQLSTALRDKFHSHVVSQQLRNASSNVNPGDGRHVGPQFFTTRTAFKRAGSSRQAIATDRFGYNSSSTCSVPQFGRTHSSTARWQVPWGSRPVGSNSYIASSQYNTGIRGASAVSFQARLQRQPDLVGVSANYAPMGPQIPSQSNAGMVGPNAVSSQHQLPRQPSFVGASVNFAPMEPVIPSQISAGIVGANAVSGQPQLPRQPSHVGVSANFALMEPEMPSHASTGLSGASAVPFQPRLPRHPRFVSASANYAPMEPRIPSQPQVSVGANSTSSESPTFVLPYAISLSSQSQVGNIYDNPLPPEAQMLSQQYAEGNSANSLPQQTSVAFHTEGGNTSVNTTASQYLLACSANSGTAPGNSSTAVFQLFNQPDGETSSNSSVPCNVDLPSPATMGSTFGSRGENTPSGQPELCRLHIPSSSDNQNISQHGYQGEDTLVPTFEDFDFDWETPISQTNSLGNVFNPKSAEPPEVSRHPQLMESITCVDIQYDDWLVSGPSDTPVSTGFNILSPEAPPIDSGFFVDF

SEQ ID NO: 21 - protein, 870 aa, unidentified
MAASPGIAEPPESSDTHSSPHPPSSPELHPTQDNPSLTTVAPSEMPNPATPVPPPPAEESTVEHSEADLLYPTPINEVEQPLSHPPQQAAPSASSAFEASPTEKLASPAPHPEEAALEVSHEVTASAPSPSSSPSPQIVGDSLEDAPQPSPPPCTPPATAAHTSTDSSSIEEIAMAYPCQEEAVRPSPMPESMDAYSRTPTAPLMPPVESRPKGLLPQQQQQPCPPSPEMAFTPCESLEPVQTLPLQPCHPVECTDASPDATVDEVVEGKLEIAASSLPVPEAGDGHKDITSLVLPALDIGSEEMLSQQQRQPLCPEMVPGRGENSKYAHPPQPPPLPESTRGWSNALTNEASAVASEEATVALQFGAERSSQEPVQTPMTSRMEPEPCSPETAPPGFEDFKSQWMPLPPPIPPAESAHNVARVAASSPVGVMCDVATESLPALEAMGVEMDTPPGLLSPLKSGAEGRSQQPLLRSCSPLVEAAPCSPDMPPPGFENCKSSWLPLPTIPPVVETTYALLDVASANTVAFMEKASFVSALESTDVETDTEQCRLSPLEGGTASSLQGPLPISPSPKMQSAPCSPDTSPPGFKKAIYVTSKEAPQSSSLDAPPSGSETGKSLPLEHTLVPSHVAEHTVCALGMVPSGSENVESSQLPQLPAVDLTLDETPDALVDAGTKTVTTEDACNPKPVTGGKEEANGSMLRPALENDGEDPLPHLEQHASSDIAPTSAEIAPTSFENSESSPQTSPCLAETDPSAQASATMLEIVKSDKTSLPLSPLQATGTDMESATLQHSPLKNEESSLAQSEQHPSSTCSCSPEVAPPGFENLESSEQLPPPPPLSAKFVVPVHFDSTIMHAPAKVISLLNYDYA

SEQ ID NO: 22 - protein, 960 aa, Vitis vinifera
MWCSSCCALPLLRTFLPSSDGCRLAGPMVPKMVTCLGKPFDIGGLNYDFIEYPLGDTFIQLNMDQKNFKVSVMTSNVRHLHDHQKLETFGNLKNSFDCYGKYSHGNTEIEKQEHESDPEINSYEAVSLTEMEIKCLNYDYVSSSDCITTDANNSKQLCSAVSTSRSSTVGVGIYKKGGAEGTSGEMVCNKVVPSSTCQNRFAGNECSNDGETSAHFIGGIDSVQQICINSGIKLETGQPLKKEVNELDLGRRKGNLLNADEDVLKSIVQATFHDKQLKTESSSSNSSHVFLPASSNDSCQEPSLDVKSCKHSKSSGMNSFCTPFKCSLEESAGKTMVLGTKAQVASREVLDSSIFLPRKQGNVGKASETVGNVSERGGEKVLGMIADLMVQRIDVPNSSLLKTKNEELASRGKLELPCEVDDGLEVASGVARETEPEVSIYREASGSSFSTEEKTGDIVNLNSIDSSYSDKNDSLIETEPRSKSYNVQVKFENLYSTKVMGEDPKLSMKNQQLCIEVKEQSQDRGLGTCQANDGLCGRGSSPLTTPKDVVGGGFDLNEDILADEVEYPKQLVNETSSSCYVVNVSAPIPVVAKSRVPLCLPMPPLQFEGQLCWKGSAATSAFRPASVSHSPNKRKALSNSDDNHSSRHSQGLKGFDLNVAAEESSLEVSPKRAERPNLDLNCLSEDDNCEASPLVSLPRNSIRDIDLNHNQWFEDTCEDAQDSGQGSQLLRGSAMDPAVSCTGNVRQPGASIARPAQPAYRADLSSKQGFSHGPAQTFLVAAPGVIPAHPFPYNKGFYFDPTNPLATICHTGVVPCMTDPHGTAVIPHALVSSTPPAFPMAPHLVNVAGGPGPCDVAIIRHSLDLNGGVGSENGSRGGNAAQLFVPVGNSLVQEQMKSFQQFALPATPIKRREPDGGWDCHQLGYRQLAAVVQSFNLIPVFAWSQLQLPQTSCDCNLRPD

SEQ ID NO: 23 - protein, 745 aa, unidentified
MSLPANGTGFCMGTVHKMTSHLCKLRNQVDDICLQVSSDSNTKGPSLSVEDKSYPANAFLPSVNVLAFLLFLVASSYFLLRKSVDTFGSCREKHAQKVSSSDSTVSNASMKNDATAAYDNLSISESSGCSHRISYMNSYKCDAYFEISDLDKACYMNSLLSLEDDDDSEWLSDLMPCGSSSEEPSTPLSYKSDAYYEISDFEQGCYIHSLLNLDDEDSEWLSDSKSFERSSEEPSTPLSYKSDSYYAILDFDKESYLHSLLSLEDEESEWLSDSKSFGCSSDNPSTPFSYKFDVASEISDLDKAVYMRSLLNFEDEDSECLSDSKSYECSSENPSTPFSCKIDLASEISDLDKARYLPSLFSFEDDDSEWLPESKSYACSSENLSTPLRHKSNAFSEISDLDKESYTHSLINLEDEDSEWFSNSKSFEYSFENPSTPLRYEGQISHLDKAGYFHSLLSLEDDDDDDDSEWLSDSKPYACSPENLSTPLSYKTDAFSEISDLDKASYMHSLLNLEGEESEWLSDSKSFGCSSSENPSTPFSYKFDDVSDISDLDKEYCMHSLLILEDENSDRLLDCKSYYECSSENSTPLSSKCDSYYNVSDLDKASYLHSLLILEDEHSEWLSDSTSWDIVVPPSVSTCSNFGDSEDKNLLETAGLEENFSADEPLFWPLEGKVNWNSEETWSSFCTSPRRRFALNSGVCSVNKKEYFALGEEVPIETLMGLKEFDGHEGLLLDSEFNDVFVVDE

SEQ ID NO: 24 - protein, 693 aa, unidentified
MVWACEERRNVDAPVRRCERLDTVGTRRGRGWPKCIGESTSLLDVIKEKTFTSPDNEADPLPNAGRVALSKKYRTARNPNSDTPKTSEYAFFKKLKKNIGPSNPDLLHRGSKLPKITQPSNCISDTTSIFNVLKYPNKDLKIPIQAEKKYKSNECLSFSPVDGVIVHQNAGISKETKLKYFSLPTAVTPVDSDLQLSPLAGPSNHSVNGHLIRLRRKHKHLFRDIYILSFLGTQYSGNGIFAAKRQKLCQRANELFLNVEKLNSERFDLVSALLKRLLPGRKEDEGFWDSQVRKGENASTSSLAYAKPDHANSRPLSRHIEDVTAPEYRMSEDDCCPSNFFGWPEERVTPELDNFTSSFHPIGFDPEAGLHYEDADNSQISNNKITINLWDQSDSSPSLSFERHGLADHFLPDRLHSAYVPVSRGAQNYFLDWDFNEEDNDPVLAISTIRGNKLYSPIATSRQVDYQQSTEKKLDALALCSSSLFTNSEYSDVQPYFCSTSFQQKFFPTDVCSKDFGMILEHEDYAVARMDRFDLPLLCYSEEQGLLEYCNTEDPFASGTAIVPYLSHHEKNHCDSDALLPMALDTFGWKFLSATSSPLQRGLSTYQSLRLPHREDTIGLTQEEIKNSLYSLNPRELAPQSVDHALNTDIWFSCNSEVSCDKAGGRSLLLANASWITSVEEISPDHCDEWTWS

SEQ ID NO: 25 - protein, 942 aa, unidentified
MGTKVQSLPGYYSMRDLNEESSSCGWPLFYGDKALANGQYYQNHLPSAAADVCSAYDKDVVKQTMLEHEAIFKNQILESRPSKVRRKMFDLHLPADEYIDTDEGEKFSDENISGTTIPDRCCKDGKGDDVNLFCGNGEKTGSHEGTSRSEQCLRSRNGLADLNEPVQVEEINAAACIPPLNHNPYQGAAECSDLSAKQKSRFFGFPTEDLLSPHRAPSNNGYLKNDGSGKLWISSKETGQGKSSSNPIPQVLKREQSFFSSTTIQDTLGKGSEPTSDYLSNRSKTGLWREKMVGGLDISQRNNAHSTDKYLESVISSHSPGLFAISPSSDFAKSRSLSAREMASSSLNQKLMSVPIPPSPFLNASGALSRSSQLHQSNGILGDGWPLNINSKHNNPNFHREAPVHPRIAEHFNNGSVNYNKRSNLICNMTSGKDINLNVRLSNGLVTQSGLGTVDGEQKHGEQLAVLPWLRSKTTCKNEAQNSGSGRHVTSGELSFLQVASLSKKDETGKGASENFMNNVTSDLCSNVNGPCMIEAGESSSKNKILGVPIFGMPLISAKESPSLTSLPVSVPNPSVTGLVENNRKNLVLDINLPCDADVLEVDMDKQSVTEIIVCKEGFSKMEATSRNQIDLNLSMSEDETVPTTIPTTNVKMKVVIDLETPAVPETEEDAILEEKQVESSSVSPLGPQVTVEQPQDEFMRYAAEAIVTMSSLCCNQVDDPASSPSETPIMDPLSWFADVASSCVDDMQRKFDNSRGKICEGKGRSSSQEMDYFESMTLQLEEIKEEDYMPQPLVPEDFILEETGTTSLPTRARKGPARRGRQRRDFQRDILPGLSSLSRHEVTEDLQTFGGLMKATGHAWNGLTRRSSSRNGFGRGRRRSQVPPSPPPPVATIETCTPLMQQLNNVEVGLEDRSLTGWGKTTRRQRRQRCPAGILPSIRLT

SEQ ID NO: 26 - protein, 688 aa, unidentified
MIRGLLILKHSCYHCLTCIFVKPCPKILIHQLYKSQGLRTLRNSCYHCLTCKFVKTYPKILIHQFYDIQGLRTLKHSCYHCLTCKFVKTCPKILIHPFCANLMVYLSVCWECNKHLQSYPHILQTIITLAHHLFLDVIWLLILDYAVGDWENKFHRDAVIQVGGSNSYASHSSKGTKSLTNTAFSVQEDVQSKNDTFQDDVLGNKSTHWRKQIANLSEAQKSVIKFVDDEKSHCKTHGSINGAITTQQEQLLDSEPKASIAIINLEEFETRQFGESLEKEVSLQHTDFPGIETRCLKGPCTGRMENGIHTANILAAESHENLKLNWESKEDFEAEINNELLLGEAFFPSQENLYERISKTKELQPNEFFLASVLSDCGAFETDGEGIADSIFDCELELDEIEESECPFDDFWTEILDVIHLEQESRDSYSADTVKVTESEVPLHIQDEDELDLLNYQEEPKDPKIANVDTSIDLDIVEDEINLPDEDSLISVDHLLSLSDHCSEVTFVGRTDRLKPNLTSDSTGHPEVAHCYEKSFLPGRNVSSSAIHRKPYPHQISSKTSCIRVNPEIPTFHPLDLYFTCSHFKNIREEKHGHYYPPAIYFYPWSKIHHESSMKTAPAMEKKYEHLERYPMLYQPDNSLQHMELLRESQADPSEHVVYDVVQQWDDMQKLPETYHQQLHYPGFGFPI

SEQ ID NO: 27 - protein, 681 aa, Sinapis alba
MEEADYEGFSIREYTRNVRSVDIRKCCPFPGEFTGDFIQSLLPSLTVTKFRWWSHELSSLLTKSPDYPKPVFGPKANAESTVPCKKRSIGETTTNDLVLHTKKIKTKKLYDSFDAKTDNKVCNCEEQARERAGDDMKCSLGIKERSGVMSPPSMSKAGVRCFSYTEEEELFPDSSKPVSQDLDSELQIPRTLKVAKRKVCSVRSLDKSSIDASQCETQVRVSLHLCCSQMNRLSLCSVAEDQSAKVMSNDKTSDNMLLESKQVNKFQPVVSERRLSHALVQSSMSCFSGSHLTLERIKDALDLERKRHVSQPNQSSISASSENHYRSCSSSLSQPASVAIHRQTLLNQSHFNPLLVEEAMSGFPLNLQGELVEANGSFRSAGAENDILLSSFVDLSSGTEPALATDVGRFTVHSVKHNKYYPARLGLDETFTENAFLVSDTNNPHTINLPKQEALFQNLSNRNMVTNSDGLCLHDTQSTMRLMGKDVSVKTGYSDVVGRGEIIASDPSMDYSFLGSYAQQSSWLWRTATLGESHSITSLDKSSKDPFPMFCEPHVGLAPQDRTAVVPDSELPSTLLHPCGSLVSCAFTDKDLYFHETGLGQQLNSVTFSQQQLPFPSNPEIAGLPSDDKNVGVVGLLPDVREQSLGLPFSCTDSTRQSLLHWPQSSFESSRLDVSYLHQSR

SEQ ID NO: 28 - protein, 684 aa, Ananas comosus
MGALSSPTQWILEDDLLLKNAMEFQQAGASLESLAKGAVSFSRRYTLKELYDRWSSILYDSNASLETSARILGFENESSTTNPSKTGRTCSSKGKDFLSHKRKVNSVRNHYYAMRKRICTEPCNTADLDFLVAPCSCAANGRQCICRGSLNSQKLHSVENIAIGVPVVNCYEQSGTTYNGGQNVFPDGIVDRDCLCRSHVNQSFEHDYIQKDPQICRENHIPLRNSFDVGETDGLQSLPSTNLYKDEIIEAKQFPICDSQIGKSEGFIVNRSEVPDSGDSFDLLGTIWKGSAPTMPMDIHITEEDPEGLTRNDTNSLQGKLNDGTADDGMNSTELISENDFIDFTGSYMEFSADDEFLFMDVDETPIVSSSPRNANQVDVANASDPKPADAIESTLVAPDLRSEGTNNSSDPIGSELDIHNATNADMEVPHTGETIEIFLICTLNTEDTEIPCNDDIILPTCESNSKHQSVSLPPSIKILSVEGKSTVGDLPIVKEENVGNTLPLTLPAKAEPSGLQHKVGLVLPSNGCTGAEFTGHPINSVNDPNSCTIHTTALKEECVAGDLGQHGNFNKSRDFFLEKPVPQVDHVNYYSANIADGPKQEIDDQIGPQKCVPANAAGDDLGLQGPIATVSTSDQEEQFSESDDDVPSYSDVEAMIIGMDLGPYDPESRPITKEGLLGDIVSR

SEQ ID NO: 29 - protein, 539 aa, Oryza sativa
MAMALALSCGRRQRGKKGGGGGGDCGGRHFHVHYHLPRRVCASSFVPNLLPRFFSLLSACVLVPPVLFLAVLAFLVLFLWFTLLYFIRSLWNKDINGETFERSSNDHGSDADKRLGEGVAEEGKAEKKTLKISSEDCARRFEIEEVLHVNSDDSQNLPSMVSSDGCLNCKMHTDDEKLIKEVTVFEIKRRESGAAIVCSDGLSQKRQPRDMSVDWFEEETKSGDIIKSGDSSPTVLSTFSSEFHDFSDNNEVVGLATDFLDVNNQNKAVLLDSSSHRGIMDNHCKCGEEFSSDKEAPAYHLFENYDFVDKHETKEVVLGDTVVLTFVDDLANDDSEYREDISEQKDPNNLSGLLDNVADIFYQQQEIHKVVVSDDNKPPEVLFLSGKQTVSSSGEFSSPNENGTSGFPFDSVCEDKNGTVAYPSVASHYNIGIEDDKCEDHTDNNIEEASSIASTNCGSADKHQTIQVLPVCLANGDNIDDVASLGSSICEDVEDKDNKSNANCISEGAPHGNGMPLVRSPASWWNLCGVIDVFAGCKD

SEQ ID NO: 30 - protein, 77 aa, unidentified
MGEEQYVETTPTLQSQPTHTLHSQIVPLSHSQPNVPIENHGVFHSSLTGDDYVCVGRSVRDMSNNIVLPSSLFREHE

SEQ ID NO: 31 - protein, 123 aa, Brassica napus
MIIEPEKMISEPEKTISEPEKMIPEPETSIPESEKMIPEPETTIPESEKMIPEPETTIPESETTVPKTNLKSICRRPRFIASSSRLSVVVVVVSPSSSPRPCRRRLSVVVVVVVSPSSSSPLR

SEQ ID NO: 32 - protein, 221 aa, Brassica sp.
MNQRPRNDYMRVNRTGKYTRKSIFDQVHTNANSIPHQPRQPQLKTQVYIIDKEDFKSIVQQLTSNQSCEFLPQNLPNRQKSRPEPTSPVPLNATGVHVSSHMGYIESLLEESSDSSGDNFQQSFDENQSHIQPQVYSNGDNFQQTFDKYQSHMQPMSYSNGPKPVMTTTLPTPWFNGSPQQIDSAYSLQSTRVEYPQPLTPNFSFSSVTQPGFFDPDLRRF

SEQ ID NO: 33 - protein, 108 aa, Brassica sp.
MMMTSGSEVVILPSPKPREATVMCLIRMSFREGPDEEQNCGHQANQESSSSIQKPDQTQDVGVMQFANQETFSSREFGPYGSSSPHFDPYREGNTWFRRTWMLAEESL

SEQ ID NO: 34 - protein, 215 aa, Brassica napus
MTEYWLASENDAQFQEVVLCHLRDNNKMVVDQPPESKNGDNDIATEQPQQGNSDDNNNRLLDFTHQQRPLIPPFEGQGLRLQTIMGYSDKATQEQQHPPISPPPQRQDSGSINNALVIMEDECVSQDEIFNLADLEAGITHPQQQHRQMMVDPYDDISFSRLAMPNNLIYHHEDSWHQDTSPWNNTNPRGLIFNSHGCEIQDQTVTKGVNQDSYY

SEQ ID NO: 35 - protein, 214 aa, Pisum sativum
MYTNEWELSEHESFEPFSIVTPLEDITTIIHETNKSFQPQQQYYQQQIEFPIWSNEFSMETTTESPFQFFQENYSNNIAKQDQPTTSFDFVVESLMSASSEDSISYSEKCSELASCSDNKPICENSPQQHKKVLEGYSALHQKSREISFQKTQLESCINQDKQCSPCGFDFSTSTNCDSKVTTRAKRRLRWTKEMHEPFVMIVNQLGGPESKFI

SEQ ID NO: 36 - protein, 188 aa, Solanum tuberosum
MIEVEENLNVEKVIVSANLENEEVVASPTKREKSKFVIMAPHQAFALVPKEGQSKQNIDQAFPKSLPHFYQSFPMQNGVGEGIKNGFKENDIVLEEMIETSVILDNEPKEQMPNWTSPSLLNPRSSCENIFKFVNIMSCHKLNEQNRVDAEESEDYNEKIMVPKHLVEEFRQFESHDKPNSEEIEIVN

SEQ ID NO: 37 - protein, 60 aa, Solanum tuberosum
MYILLQAVFTIATTALTVPIFLSYKLHVVFQIFKVSASIWNGGNFMLDVMPRQVILKEEK

SEQ ID NO: 38 - protein, 59 aa, Solanum tuberosum
MDRHVILFLRCPWRRYVEVGNAEVDGENGVVEQDKYSIRPAHTISETEKNQITSSWRLS

SEQ ID NO: 39 - protein, 173 aa, Solanum tuberosum
MALPHHHLQLHIQQQQPHQQQQQQQSKSYRDLYNNMDGQITTPVVYFNGSNLPEQSQHPPYIPPFQVVGLAPGTADDGGLDLQWNYGLEPKKKRPKEQDFMENNNSQISSVDLLQRRSVSTGLGLSLDNGRLASSCDSAFLGLVGDDIERELQRQDAEIDRYIKVQVFTPSID

SEQ ID NO: 40 - protein, 100 aa, Solanum tuberosum
MWQLEAAARSAIENGMEEGHGDSTDEFVPIAFPQAMHMEMEANNVPVRNNNPTITALRRYEDQIVPSMSSSFFRNEDEETSVDIRNSVVDSRAEVPIISM

SEQ ID NO: 41 - protein, 116 aa, unidentified
WSKIAKHLPGRTDNEIKNYWRTRIQKHIKQAENSQQQSSSDIQINDNDNQIGSTSQISAMPEPMDINIISPPSYQGMLEPFPNQFPTISDQSSCCTNDNNYWSMEDLLSLQLLNGD

SEQ ID NO: 42 - protein, 108 aa, unidentified
MTRAYSVKGKKRKNKDVVEKYHREDKEVADEEQVQPKKPNLQKEEEPTQTPTPKITEENNNNELVGIPIAPPTENNSEKPGVIFILEKASLEVAKVGKVLQFSALFLL

SEQ ID NO: 43 - protein, 156 aa, unidentified
MTNETTETTTMVTSQQVVEVSEPSKKPIIPFFPNFNFNFQIPQFPFPQFLPKNHRHDDAGGADKNNETPQLHSEGGQPGPNVVTFPKSQQVVVPSPLQAEADANSSTAKTSHPIVIYQVYAIGAFFLSQWIWARWNERKARGGSPDDEGRGPQDNK

SEQ ID NO: 44 - protein, 29 aa, unidentified
MKHHSSHHGHHGHHGHHGHHGHHGHHGHH

SEQ ID NO: 45 - protein, 50 aa, unidentified
MKHHSSHHGHHGHHGHHGHHGHHGHHGHHGTYPYDVPDYAGTSENLYFQG

SEQ ID NO: 46 - protein, 129 aa, unidentified
MKHHSSHHGHHGHHGHHGHHGHHGHHGHHGTSAAGGEEDKKPAGGEGGGAHINLKVKGQDGNEVFFRIKRSTQLKKLMNAYCDRQSVDMTAIAFLFDGRRLRAEQTPDELEMEDGDEIDAMLHQTGGGS

SEQ ID NO: 47 - DNA, 87 bp, unidentified
  source 1..87: mol_type = other DNA
atgaaacatcatagtagtcaccacggtcatcacggtcatcacggtcatcacggtcatcacgggcatcacggtcatcacggtcatcac

SEQ ID NO: 48 - DNA, 150 bp, unidentified
  source 1..150: mol_type = other DNA
atgaaacatcatagtagtcaccacggtcatcacggtcatcacggtcatcacggtcatcacgggcatcacggtcatcacggtcatcacggtacctacccttacgacgtgccagactacgccggaactagtgagaacctgtattttcaagga

SEQ ID NO: 49 - DNA, 387 bp, unidentified
  source 1..387: mol_type = other DNA
atgaaacatcatagtagtcaccacggtcatcacggtcatcacggtcatcacggtcatcacgggcatcacggtcatcacggtcatcacggtacctctgctgcgggtggcgaagaagataagaaaccggcaggtggcgaaggtggcggtgcccatatcaacctgaaagtgaaaggtcaagacggcaacgaagtctttttccgcatcaaacgttctacccagctgaaaaagctgatgaacgcatactgtgaccgtcagtctgtagacatgaccgcaattgctttcctgtttgatggtcgtcgcctgcgtgcggaacagaccccggatgaactggagatggaagatggcgacgaaatcgacgcaatgctgcaccagactggtggcggatcc